(19) United States
(12) Patent Application Publication
(10) Pub. No.: US 2018/0055887 A1
(43) Pub. Date: Mar. 1, 2018

(54) METHOD FOR PREPARING INDUCED MESENCHYMAL STEM CELLS AND IMPROVING MESENCHYMAL STEM CELL'S CHARACTERS AND ITS APPLICATIONS

(71) Applicant: ACADEMIA SINICA, Taipei City (TW)

(72) Inventors: Jean LU, Taipei City (TW); Hsiao-Chun HUANG, Taipei City (TW); Pei-Lun LAI, New Taipei City (TW)

(73) Assignee: ACADEMIA SINICA, Taipei City (TW)

(21) Appl. No.: 15/684,207

(22) Filed: Aug. 23, 2017

Related U.S. Application Data
(60) Provisional application No. 62/378,556, filed on Aug. 23, 2016.

(51) Int. Cl. A61K 35/28; C12N 5/0775

(52) U.S. Cl. CPC A61K 35/28; C12N 5/0663; A61K 2035/124; C12N 5/0664; C12N 5/0667

(57) ABSTRACT

The present invention generally relates to a method for preparing induced mesenchymal stem cells (iMSCs) and its applications. The iMSCs, like MSCs, can differentiate into multiple lineages, which may be beneficial for disease treatments. In addition, the present invention also provides a method for improving the MSC's functional characteristics such that the MSCs are more suitable for cell therapy or in vitro applications.

### RELATED APPLICATIONS

This application claims the benefit of U.S. provisional application No. 62/378,556, filed Aug. 23, 2016 under 35 U.S.C. §119, the entire content of which is incorporated herein by reference.

### TECHNOLOGY FIELD

The present invention generally relates to a method for preparing induced mesenchymal stem cells or improving MSC characters and their applications.

### BACKGROUND OF THE INVENTION

Mesenchymal stromal/stem cells (MSCs) can self-renew and are multipotent. They were first isolated from bone marrow and can differentiate into multiple lineages, including bone, fat, cartilage, hepatocytes, neurons, islet cells, fibroblasts, etc^{1,2}. In addition, MSCs constitute essential niche to maintain hematopoietic stem cells and other adult stem cells³. MSCs are multipotent, exhibit immunoregulatory functions⁴, and secrete multiple cytokines that promote tissue healing⁵. Thus MSCs hold great promise for the treatment of multiple diseases. Most importantly, unlike embryonic stem cells (ESCs) or induced pluripotent stem cells (iPSCs) which are oncogenic, MSCs do not have oncogenic ability and therefore are considered to have greater biosafety¹⁴.

Many clinical trials registered in ClinicalTrials.gov (www.clinicaltrials.gov) use MSCs for disease treatments. The trials include acute lung injury (ALI)⁶ graft-versus-host Disease⁷, Crohn's disease⁸, type 1 diabetes mellitus⁹, diabetic wounds, multiple sclerosis, neurological diseases (spinal cord injury, Parkinson's disease, Alzheimer's disease, amyotrophic lateral sclerosis, diabetic peripheral neuropathy, epilepsy, schizophrenia, autism)¹⁰, cardiovascular diseases (myocardial infraction, ischemic heart disease, chronic heart failure, coronary artery disease, dilated cardiomyopathy peripheral vascular diseases, non-ischemic dilated cardiomyopathy)¹¹, osteogenesis imperfecta¹², ulcerative colitis, stem cell engraftment, cirrhosis, fractures, cartilage injury, kidney transplant, renal failure, osteoarthritis, acute respiratory distress syndrome, Sjögren's syndrome (pSS), systematic sclerdomerma, Duchenne muscular dystrophy, cancers, degenerative disc disease, arthroscopic rotator cuff repair, anemia, critical limb ischemia, neuromyelitis optica spectrum disorders, subclinical rejection of organ tranpinatation, maxillary cyst, atherosclerosis, premature ovarian failure, anterior cruciate ligament injury, articular chondral defect, Kienböck's disease, sepeis/septic shock, perianal fistula, osteonecrosis, pseudoarthrosis, delayed graft function, focal segmental glomerulosclerosis, chronic obstructive pulmonary disease, osteochondritis, rheumatoid arthritis, dysphonia, osteonecrosis, drug-induced neutropenia, brain injuries, burn wound, acute kidney injury, breast reconstruction, liver failure, liver cirrhosis, foreign body reaction, inflammation, effusion (L) knee, skin ulcer, recto-vaginal fistula, dystrophic epidermolysis bullosa, osteoporosis, local feminine stress urinary incontinence treatment (HULPURO), retinal disease, macular degeneration, hereditary retinal dystrophy, optic nerve disease, glaucoma, hip arthroplasty, cerebral palsy, male infertility, arthrodesis, Romberg's disease, ankylosing spondylitis, uremia, chronic meniscal injury, cutaneous photoaging, emphysema, bronchopulmonary dysplasia, fecallncontinence, idiopathic pulmonary fibrosis, autoimmune hepatitis, biliary cirrhosis, spondyloarthrosis, epidermolysis bullosa, asthma, xerostomia, dementia, recovery of medial meniscectomy, progressive supranuclear palsy, psoriasis vulgaris, CMV infection, rotator cuff disease, cytopenia, myelodysplastic syndromes, Peyronie's Disease, limbus corneae insufficiency syndrome, Romberg's disease, liver regeneration, refractory systemic lupus erythematosus, ulcerative colitis, paraquat Poisoning, pneumonia, emphysema, aging frailty, lung transplantation, bone cyst, cerebral adrenoleukodystrophy, erectile dysfunction, intervertebral disc disease, lipodystrophies, Buerger's disease, hemophilia, Wilson's disease, bronchiectasis, retinitis pigmentosa, cerebellar Ataxia, sweat gland diseases, systemic lupus erythematosus, Devic's Syndrome, cleft lip and palate, Sjogren's Syndrome and Hurler's syndrome¹³. Currently, about 543 clinical trials are examining the efficacy of MSCs in cell therapy (www.clinicaltrials.gov).

MSCs also have been approved to treat graft-versus-host disease in Canada and New Zealand and degenerative arthritis and anal fistula in Korea¹⁵. In addition, MSCs already shown beneficial effects in the clinical trials of diabetics, multiple sclerosis, kidney transplantation, Crohn's disease, systemic lupus erythematosus (SLE), and ulcerative colitis¹⁵ (www.clinicaltrials.gov). Till now, almost no safety concern has been reported in MSCs in the clinical trials¹⁶.

MSCs also hold great promise to treat additional diseases due to its ability to differentiate into multiple cell types. MSCs can differentiate into osteoblasts (bone), chondrocytes (cartilage), adipocytes (fat), neurons, hepatocytes, β cells, etc. The MSC-derived cells might be used in the clinic for tissue engineering and regenerative medicine. For example, MSCs may be applicable in cartilage and bone regeneration for the treatments of arthritis, lower back pain (LBP), cartilage degeneration, bone fracture, or osteoporosis. The diseases that can be treated by MSC-derived cells include but not limited to diabetes, neurodegenerative diseases (e.g. Parkinson, Alzheimer diseases and amyotrophic lateral sclerosis), liver Diseases (e.g. hepatitis, alcohol abuse) and liver transplantation. In addition, since MSCs can differentiate into fat and cartilage, MSCs may also be applicable in plastic surgery such as autologous fat transplantation and cartilage grafting in nasal augmentation. MSCs also can support the hematopoietic stem cells and other adult stem cells engraftment or maintenance³.

MSCs can be obtained from various sources, such as bone marrow, adipose or dental tissues and then cultured for expansion. Clinically, the preferred source is bone marrow aspirated from the iliac crest or adipose tissue, which needs an invasive and painful surgical procedure for patients. ESCs or iPSCs can differentiate to MSCs; however such manner involves oncogenic risks. In addition, it has been reported that platelet-derived growth factor-AB (PDGF-AB) and 5-Azacytidine (AZA) are effective in inducing conversion of mouse osteocytes and human adipocytes into MSC-like cells²⁶. However, these conversion protocols require the use of fetal bovine serum (FBS) and take 25 days to accomplish the conversion which is time consuming. In addition, obtaining human adipocytes need liposuction, it is not as easy as skin puncture. Use of FBS is disadvantageous for cell therapy in subjects due to unknown components in the FBS and concerns of animal products, which increases infectious risks and other problems. There is still a need to develop an improved method of producing induced MSCs (iMSCs) from somatic cells, particularly using small molecules, without need of animal components, in a more efficient manner.

In addition, it is known that primary isolated MSCs from some donors (like aging) are of a low level of MSC's functional characteristics, in particular the activities in expansion, clonogenicity and/or differentiation and thus are not perfect for cell therapy. Some growth factors are reported to improve the above MSC's functional characteristics. Until now, there are no published papers describing a method of enhancing MSC's functional characteristics using non-protein small molecules.

### SUMMARY OF THE INVENTION

In this invention, it is unexpectedly found that induced MSCs (iMSCs) can be successfully generated by culturing skin cells e.g. fibroblasts in a culture medium which comprises at least a protein kinase C (PKC) inhibitor and/or a glycogen synthase kinase 3 beta (GSK3β) inhibitor. According to the present invention, skin cells e.g. fibroblasts can be dedifferentiated/reprogrammed into iMSCs which can differentiate into multiple lineages and beneficial for disease treatment.

Therefore, in one aspect, the present invention provides a method of generating induced mesenchymal stem cells (iMSCs), comprising culturing skin cells e.g. fibroblasts in a condition which allows a proportion of the skin cells to dedifferentiate/reprogram into iMSCs, wherein the condition comprises a culture medium which comprises a protein kinase C (PKC) inhibitor and/or a glycogen synthase kinase 3 beta (GSK3β) inhibitor.

In some embodiments, the culture medium further comprises an auxiliary agent to enhance the efficacy of dedifferentiation/reprogramming from the skin cells to iMSCs, which is selected from the group consisting of a p38 inhibitor (e.g. SB202190 or SB203580), a c-jun N terminal kinase (JNK) inhibitor (e.g. SP600125), a Rho-associated protein kinase (ROCK) inhibitor (e.g. Y-27632), an extracellular regulated kinase (ERK) inhibitor (e.g. PD325901), a AMP-activated protein kinase (AMPK) inhibitor (e.g. Dorsomorphin), a bone morphogenesis protein inhibitor (e.g. Dorsomorphin), a Src tyrosine kinase inhibitor (e.g. PP1, Dasatinib), an anaplastic lymphoma kinase (ALK) inhibitor (e.g. SB431542), a phosphoinositide 3-kinase inhibitor (PI3K) inhibitor (e.g. LY294002), a cyclic adenosine monophosphate (cAMP) activator (e.g. Froskolin, Rolipram), a histone deacetylase (HDAC) inhibitor (e.g. VPA), an antioxidant (e.g. NAC, GSH, Vitamin C. etc.), a tumor growth factor beta (TGFβ) inhibitor (e.g. Repsox), a target of rapamycin (mTOR) inhibitor (e.g. Rapamycin), a G9a methyltransferase inhibitor (e.g. BIOX01294), a DOTIL inhibitor (e.g. SGC0946), and any combination thereof.

Specifically, PKC inhibitors, GSK3β inhibitors and auxiliary agents as used herein are small molecules.

In some embodiments, the culture medium used in the present invention comprises a combination selected from the group consisting of:

(1) a combination of a PKC inhibitor and a ROCK inhibitor;
(2) a combination of a PKC inhibitor, a ALK inhibitor and a ROCK inhibitor;
(3) a combination of a PKC inhibitor and a Src family tyrosine kinase inhibitor;
(4) a combination of a PKC inhibitor and a GSK3β inhibitor;
(5) a combination of a PKC inhibitor and a HDAC inhibitor;
(6) a combination of a PKC inhibitor, a HDAC inhibitor and a Src tyrosine kinase inhibitor;
(7) a combination of a PKC inhibitor, a HDAC inhibitor and a target of rapamycin (mTOR) inhibitor;
(8) a combination of a PKC inhibitor and a cAMP activator;
(9) a combination of a PKC inhibitor, a HDAC inhibitor and a G9a methyltransferase inhibitor;
(10) a combination of a PKC inhibitor, a HDAC inhibitor and a DOT1L inhibitor;
(11) a combination of a PKC inhibitor, a HDAC inhibitor, a JNK inhibitor and a p38 inhibitor;
(12) a combination of a PKC inhibitor, a GSK3β inhibitor, a JNK inhibitor, a p38 inhibitor, a ROCK inhibitor and a ERK inhibitor;
(13) a combination of a PKC inhibitor, a HDAC inhibitor and a cAMP activator;
(14) a combination of a PKC inhibitor and a AMPK inhibitor/BMP inhibitor;
(15) a combination of a PKC inhibitor, a GSK3β inhibitor and a HDAC inhibitor;
(16) a combination of a PKC inhibitor, a GSK3β inhibitor, a HDAC inhibitor, a ROCK inhibitor and an ERK inhibitor;
(17) a combination of a PKC inhibitor, a HDAC inhibitor and a AMPK inhibitor BMP inhibitor;
(18) a combination of a PKC inhibitor, a GSK3β inhibitor, a HDAC inhibitor and a AMPK inhibitor BMP inhibitor;
(19) a combination of a PKC inhibitor, a GSK3β inhibitor, a HDAC inhibitor, a JNK inhibitor, a p38 inhibitor, a ROCK inhibitor and a ERK inhibitor; and
(20) a combination of a PKC inhibitor, a GSK3β inhibitor, a HDAC inhibitor, a JNK inhibitor, a p38 inhibitor, a ROCK inhibitor, a ERK inhibitor, and a AMPK inhibitor/BMP inhibitor.

In some embodiments, the skin cells are fibroblasts, preferably obtained from human cells.

In some embodiments, the skin cells are cultured in the culture medium for at least 1 day or more (e.g. 2 days, 3 days, 4 days, 5 days, 6 days, 7 days, 8 days, 9 days, 10 days or more).

In some embodiments, at least 0.9%, 1%, 5%, 10%, 15%, 20%, 25%, 30%, 35%, 40%, 45%, 50%, 55%, 60%, 65%, 70%, 75%, 80% or more (e.g. about 80%) of the skin cells are dedifferentiated/reprogrammed into iMSCs.

In some embodiments, the skin cells are fibroblasts including neonatal fibroblasts or adult fibroblasts.

In some embodiments, the culture medium is serum free.

In some embodiments, the iMSCs which are dedifferentiated/reprogrammed from the skin cells have one or more features selected from the group consisting of: (i) the iMSCs can be maintained and expanded for at least 3 cultivation passages, (ii) the iMSCs are multipotent, (iii) the iMSCs express a MSC marker, and any combination of the above.

In some embodiments, the iMSCs express a MSC marker selected from the group consisting of stage-specific embryonic antigen (SSEA)-4, podocalyxin-like protein (PODXL) and a combination thereof.

In some embodiments, the iMSCs further express a MSC marker selected from the group consisting CD105, CD73, CD44, CD90 and a combination thereof.

In some embodiments, the iMSCs are negative for CD45, CD34, CD11b, CD19.

In some embodiments, the iMSCs are SSEA-4⁺, PODXL⁺, CD105⁺, CD73⁺, CD44⁺, CD90⁺, CD45⁻, CD34⁻, CD11b⁻, CD19⁻.

In some embodiments, the method of the invention further comprises isolating cells expressing a MSC marker to obtain an isolated iMSC population.

In another aspect, the present invention provides a cell culture comprising iMSCs as described herein. Specifically, comparing to the nature BMMSCs, which express the functional markers, SSEA-4 and PODXL, from about 35% to about 50%, the cell culture of the present invention include 0.9%% to 80% of iMSCs, particularly 50% or higher, 55% or higher, 60% or higher, 65% or higher, 70% or higher, 75% or higher, 80% or higher of MSCs after chemical induction as described herein.

In a further aspect, the present invention provides an isolated population of iMSCs as described herein.

Also provided is a pharmaceutical composition comprising iMSCs as generated by the above-described method.

In still a further aspect, the present invention provides a method of producing differentiated somatic cells, comprising subjecting iMSCs as described herein to a condition suitable for differentiation, thereby producing specific somatic cells. Specifically, the iMSCs are derived from skin cells via treatment with a protein kinase C (PKC) inhibitor and/or a glycogen synthase kinase 3 beta (GSK3β) inhibitor, and optionally one or more auxiliary agents as described herein.

In some embodiments, the specific somatic cells differentiated from the iMSCs are selected from the group consisting of fibroblasts, adipocytes, chondrocytes, osteoblasts, osteocytes, myoblasts, neurons, beta islet cells, hepatocytes, cardiomyocytes, and neural stem cells.

In still an additional aspect, the present invention provides a method for treating a disease or disorder, comprising administering a therapeutically effective amount of iMSCs as described herein to a subject in need of such treatment. Specifically, the iMSCs are derived from skin cells via treatment with a protein kinase C (PKC) inhibitor and/or a glycogen synthase kinase 3 beta (GSK3β) inhibitor, and optionally one or more auxiliary agents as described herein. Also provided is use of iMSCs as described herein in the manufacture of a medicament for treating a disease or disorder.

In some embodiments, the disease or disorder to be treated according to the present invention is selected from the group consisting of acute lung injury (ALI), graft-versus-host Disease, Crohn's disease, type 1 diabetes mellitus, diabetic wounds, multiple sclerosis, neurological diseases (spinal cord Injury, Parkinson's disease, Alzheimer's disease, amyotrophic lateral sclerosis, diabetic peripheral neuropathy, epilepsy, schizophrenia, autism), cardiovascular diseases (myocardial infraction, ischemic heart disease, chronic heart failure, coronary artery disease, dilated cardiomyopathy peripheral vascular diseases, non-ischemic dilated cardiomyopathy), osteogenesis imperfecta, ulcerative colitis, stem cell engraftment, cirrhosis, fractures, cartilage injury, kidney transplant, renal failure, osteoarthritis, acute respiratory distress syndrome, Sjögren's syndrome (pSS), systematic sclerdomerma, Duchenne muscular dystrophy, cancers, degenerative disc disease, arthroscopic rotator cuff repair, anemia, critical limb ischemia, neuromyelitis optica spectrum disorders, subclinical rejection of organ transplantation, maxillary cyst, atherosclerosis, premature ovarian failure, anterior cruciate ligament injury, articular chondral defect, Kienböck's disease, sepeis/septic shock, perianal fistula, osteonecrosis, pseudoarthrosis, delayed graft function, focal segmental glomerulosclerosis, chronic obstructive pulmonary disease, osteochondritis, rheumatoid arthritis, dysphonia, osteonecrosis, drug-induced neutropenia, brain injuries, burn wound, acute kidney injury, breast reconstruction, liver failure, liver cirrhosis, foreign body reaction, inflammation, effusion (L) knee, skin ulcer, recto-vaginal fistula, dystrophic epidermolysis bullosa, osteoporosis, local feminine stress urinary incontinence treatment (HULPURO), retinal disease, macular degeneration, hereditary retinal dystrophy, optic nerve disease, glaucoma, hip arthroplasty, cerebral palsy, male infertility, arthrodesis, Romberg's disease, ankylosing spondylitis, uremia, chronic meniscal injury, cutaneous photoaging, emphysema, bronchopulmonary dysplasia, fecallncontinence, idiopathic pulmonary fibrosis, autoimmune hepatitis, biliary cirrhosis, spondyloarthrosis, epidermolysis bullosa, asthma, xerostomia, dementia, recovery of medial meniscectomy, progressive supranuclear palsy, psoriasis vulgaris, CMV infection, rotator cuff disease, cytopenia, myelodysplastic syndromes, Peyronie's Disease, limbus corneae insufficiency syndrome, Romberg's disease, liver regeneration, refractory-systemic lupus erythematosus, ulcerative colitis, paraquat-Poisoning, pneumonia, emphysema, aging frailty, lung transplantation, bone cyst, cerebral adrenoleukodystrophy, erectile-dysfunction, intervertebral disc disease, lipodystrophies, Buerger's disease, hemophilia, Wilson's disease, bronchiectasis, retinitis pigmentosa, cerebellar Ataxia, sweat-gland-diseases, systemic lupus erythematosus, Devic's Syndrome, cleft-lip-and-palate, Sjogren's-Syndrome and Hurler's syndrome.

In this invention, it is further found that the chemical agent(s) as described herein can be used to enhance the MSC's functional characteristics, in particular the activities in expansion, clonogenicity and/or differentiation.

Therefore, in another aspect, the present invention provides a method of improving functional characteristics of MSCs, comprising treating the MSCs with one or more chemical agent(s) as described herein. The functional characteristics of MSCs include but are not limited to expansion, clonogenicity and/or differentiation.

The details of one or more embodiments of the invention are set forth in the description below. Other features or advantages of the present invention will be apparent from the following detailed description of several embodiments, and also from the appending claims.

### BRIEF DESCRIPTION OF THE DRAWINGS

The foregoing summary, as well as the following detailed description of the invention, will be better understood when read in conjunction with the appended drawings. For the purpose of illustrating the invention, there are shown in the drawings embodiments which are presently preferred. It should be understood, however, that the invention is not limited to the precise arrangements and instrumentalities shown.

In the drawings:

FIGS. 1A, 1B, 1C, 1D, 1E and 1F include charts showing derivation of iMSCs from neonatal and adult fibroblasts with chemical cocktails and growth factors.

FIG. 1A shows an experimental scheme for efficient chemical-based derivation of iMSCs from dermal fibroblasts. Expanded iMSCs can be further differentiated into different lineages or treat disease in the mouse model. FIG. 1B shows a representative flow cytometry analysis of human neonatal fibroblasts (CRL2097), iMSCs, and BMMSCs with MSC markers, SSEA-4 and PODXL. SSEA-4 and PODXL were abundantly expressed in iMSCs and BMMSCs. FIG. 1C shows consistent production of iMSCs from neonatal fibroblasts with high efficiency. The chemical cocktail reproducibly converts human fibroblasts into iMSCs with high efficiency. Human fibroblasts were untreated (fibroblasts) or treated with the chemical cocktail [6 Chemical (6C) that includes+3 GF; p38 inhibitor (SB202190, SB203580), JNK inhibitor (SP600125), PKC inhibitor (Go6983), ROCK inhibitor (Y-27632), ERK1/2 inhibitor (PD0325901), GSK3β inhibitor (CHIR99021), and three growth factors (3GF) which include human LIF, bFGF, TGF-β)]) for 6 days and then subjected to flow cytometry analysis with MSC markers, SSEA-4 and PODXL. Ten independent experiments were performed, and the average iMSC induction rate of the chemical cocktail (6C+3 GF) was shown to be 37.62%. FIG. 1D shows that iMSCs derived from neonatal fibroblasts express tradition MSC surface markers defined by ISCT. Identity of mesenchymal stem cells by the markers of ISCT's proclamation. Traditional MSC markers were examined by flow cytometry with indicated antibody or isotype control. BMMSCs served as the control. iMSCs express CD90, CD44, CD73 and CD105 and do not express CD11 b, CD19, CD34, CD45 and HLA-DR. FIG. 1E shows hierarchical clustering of gene expression profiles for neonatal fibroblasts (CRL2097), iMSCs induced from neonatal (CRL2097) and adult dermal fibroblasts (42 and 56 year-old females), and two different BMMSCs (BMMSC_1: primary human bone marrow MSCs used throughout this study; BMMSC_2: publicly available gene expression data for human BMMSCs with accession number GSM1533333). FIG. 1F shows a principal component analysis of the expression of stemness genes for fibroblasts (CRL2097, 42 and 56 year-old females), iMSCs induced from these three fibroblasts, and two independent sources of BMMSCs (BMMSC_1 and BMMSC_2). Principal component 1 accounts for 40%, principal component 2 accounts for 19%, and principal component 3 accounts for 14% of the variation of the dataset. Clustering of iMSCs derived from three different fibroblast sources suggests the robust efficacy of the cocktail.

FIG. 2 shows that iMSCs can expand in MSC cultured medium for at least 8 passages. iMSCs cultured for 8 passages after sorting stably express SSEA-4 and PODXL. Foreskin neonatal fibroblasts CRL2097 were treated with the chemical cocktail (6C+3 GF) and sorted using SSEA-4 and PODXL antibodies. The resulting iMSCs were cultured in regular MSC medium (DMEM-LG+10% FBS) for 8 passages. Representative immunofluorescent images of BMMSCs, iMSCs (at passage 8), and neonatal fibroblasts (fibroblasts) using antibodies against SSEA-4 and PODXL are shown. Scale bar, 50 μm.

FIGS. 3A, 3B and 3C include charts showing that iMSCs derived from neonatal fibroblasts are multipotent and differentiation ability is comparable to BMMSCS. FIG. 3A shows early osteogenesis. Neonatal fibroblasts (CRL2097), iMSCs derived from CRL2097 (iMSCs), and BMMSCs were cultured in osteogenic induction medium for 10 days, and the alkaline phosphatase (ALP) activity assay was performed. The quantification data is shown in the lower panel. The ALP amounts of iMSCs are comparable to BMMSCs, while the ALP amounts of fibroblasts are barely detectable. FIG. 3B shows late osteogenesis. Alizarin Red S staining (ARS) was performed at day 21. The quantification data is shown in the lower panel. The ARS amounts of iMSCs are comparable to BMMSCs, while the ARS amounts of fibroblasts are barely detectable. FIG. 3C shows adipogenesis. Neonatal fibroblasts, iMSCs, and BMMSCs were cultured in adipogenic induction medium for 21 days and then stained with Oil Red O. The quantification data is shown in the lower panel. The oil red amounts of iMSCs are comparable to BMMSCs, while the oil red amounts of fibroblasts are barely detectable. Scale bar, 50 μm. n=6 for all samples. ****p<0.0001. FIG. 3D shows chondrogenesis. Lacunae structure (Hematoxylin and eosin staining, HE) and proteoglycans of cartilage (Alcian Blue staining) were examined to evaluate the ability of iMSCs to differentiate into chondrocytes at day 21. Lacunae structure is marked by the yellow arrow. Scale bar, 50 μm. iMSCs differentiate into chondrocytes to a degree comparable to BMMSCs.

FIGS. 4A, 4B and 4C include charts showing that iMSCs derived from adult fibroblasts are multipotent. FIG. 4A shows osteogenesis. The iMSCs derived from human adult fibroblasts (42 and 56 year-old females) exhibit osteogenesis abilities comparable to those of BMMSCs. Indicated fibroblasts (42 and 56 year-old females), iMSCs derived from adult fibroblasts (42 and 56 year-old females), and BMMSCs were cultured in osteoblast-induction medium for 21 days, and were then assayed by Alizarin Red staining (ARS) (upper panel). The dye was extracted and ARS was quantified by measuring the optical density (O.D.) at 550 nm (lower panel) (n=6). ****p<0.0001. FIG. 4B shows adipogenesis. The iMSCs derived from human adult fibroblasts (42 and 56 year-old females) exhibit adipogenesis abilities comparable to those of BMMSCs. Indicated fibroblasts, iMSCs, and BMMSCs were cultured in adipocyte induction medium for 21 days, and the lipid drops were then stained with Oil Red O (upper panel). Scale bar, 50 μm. The dye was extracted, and Oil Red O staining was quantified by measuring the O.D. at 530 nm (lower panel) (n=6). ****p<0.0001. FIG. 4C shows chondrogenesis. The iMSCs derived from human adult fibroblasts (42 and 56 year-old females) exhibit chondrogenesis abilities comparable to those of BMMSCs. Lacunae structure (revealed by hematoxylin and eosin staining, HE stain, upper panel) (marked by yellow arrow) and proteoglycans of cartilage (revealed by Alcian Blue staining, lower panel) were examined to evaluate the capacity of cells to differentiate into chondrocytes at day 21. Three independent experiments were performed. Scale bar, 100 μm.

FIGS. 5A, 5B and 5C include charts showing that iMSCs derived from neonatal fibroblasts markedly decrease the fatality of endotoxin-induced acute lung injury in the mouse model. Neonatal fibroblasts (CRL2097), iMSCs derived from CRL2097 (iMSCs), and BMMSCs were injected into mice 4 hours post the administration of liposaccharides (LPS, an endotoxin). Results were analyzed 48 hours post injection. FIG. 5A shows the histology of lung. iMSCs and BMMSCs ameliorate the lung inflammation. Representative lung histology at 48 h after LPS-induced acute lung injury. Scale bar, 100 m. FIG. 5B shows the survival curve. iMSCs, to a degree comparable to BMMSCs, efficiently improve the survival rate of LPS-induced acute lung injury. Results are expressed as percentage survival (n=10-12 per group). *p<0.05, LPS+PBS V.S. PBS, LPS+iMSCs, or LPS+BMMSCs. ^{#}p<0.05, LPS+Fibroblasts V.S. PBS, LPS+iMSCs, or LPS+BMMSCs. FIG. 5C shows the injury score of the lung. Injury score of LPS-induced acute lung injury. Quantification of histology at 48 h after LPS-induced acute lung injury, all sections were quantified after digital slide scanning of the whole slide, n=10-12 each group. Injury score are using the following criteria: 0, no injury; 1, 25% injury in the field; 2, 50% injury in the field; 3, 75% injury in the field; 4, diffuse lung injury. * p<0.05.

FIG. 6 shows growth factors are dispensable for the conversion of human fibroblasts into iMSCs. Fibroblasts were treated with four or six chemicals combined with the indicated growth factors, and then subjected to flow cytometry analysis at day 6 to quantify iMSC conversion efficiency (SSEA-4⁺PODXL⁺ population). The addition of growth factors does not promote iMSC conversion of cells treated with the four or six chemical cocktail.

FIGS. 7A and 7B include charts showing that the iMSCs derived from only six chemicals are multipotent. FIG. 7A shows osteogenesis. The iMSCs derived from human neonatal fibroblasts exhibit osteogenesis abilities comparable to those of BMMSCs. iMSCs derived from neonatal fibroblasts, and BMMSCs were cultured in osteoblast-induction medium for 21 days, and were then assayed by Alizarin Red staining (ARS) (left panel). The dye was extracted and ARS was quantified by measuring the optical density (O.D.) at 550 nm (right panel) (n=6). ****p<0.0001. FIG. 7B shows adipogenesis. The iMSCs derived from human neonatal fibroblasts exhibit adipogenesis abilities comparable to those of BMMSCs. Indicated fibroblasts, iMSCs, and BMMSCs were cultured in adipocyte induction medium for 21 days, and the lipid drops were then stained with Oil Red O (left panel). Scale bar, 50 μm. The dye was extracted, and Oil Red O staining was quantified by measuring the O.D. at 530 nm (right panel) (n=6).

FIGS. 8A and 8B include charts showing that chemical treatment can enhance multipotency of MSCs. FIG. 8A shows treatment with six chemicals with three growth factors (6C+3GF) increases the expressions of SSEA4 and PODXL functional markers. FIG. 8B shows treatment with six chemicals (6C), seven chemicals (7C) and eight chemicals (8C) increases the expressions of SSEA4 and PODXL functional markers. [6C: a p38 inhibitor (SB202190), a JNK inhibitor (SP600125), a protein kinase C inhibitor (Go6983), a ROCK inhibitor (Y-27632), a ERK1/2 inhibitor (PD0325901) and a GSK3β inhibitor (CHIR99021). 7C: a p38 inhibitor (SB202190), a JNK inhibitor (SP600125), a protein kinase C inhibitor (Go6983), a ROCK inhibitor (Y-27632), a ERK1/2 inhibitor (PD0325901) a GSK3β inhibitor (CHIR99021), and a HDAC inhibitor (VPA). 8C: a p38 inhibitor (SB202190,), a JNK inhibitor (SP600125), a protein kinase C inhibitor (Go6983), a ROCK inhibitor (Y-27632), a ERK1/2 inhibitor (PD0325901), a GSK3β inhibitor (CHIR99021), a HDAC inhibitor (VPA), and a BMPa AMPK/BMP inhibitor (Dorsomorphin)].

FIGS. 9A and 9B include charts showing rejuvenation of aging MSCs by optimized cocktails 6C, 7C or 8C with stronger multipotency. FIG. 9A shows the morphology of the aging MSCs derived from 40 and 69 year-old men. FIG. 9B shows that after pre-treated with chemical cocktail 6C, 7C, and 8C for 6 days, the rejuvenated MSCs were cultured in regular MSC medium for 3 days then switched to osteogenic medium for 7 days for ALP test. The image shows the osteogenic levels increase after the treatment of chemical cocktails. The quantification results of ALP were examined using one-way ANOVA complemented with Tukey's test, n=3, *p<0.05. Human aging MSCs were untreated or treated with the chemical cocktail [6C: a p38 inhibitor (SB202190), a JNK inhibitor (SP600125), a protein kinase C inhibitor (Go6983), a ROCK inhibitor (Y-27632), a ERK1/2 inhibitor (PD0325901) and a GSK3β inhibitor (CHIR99021). 7C: a p38 inhibitor (SB202190), a JNK inhibitor (SP600125), a protein kinase C inhibitor (Go6983), a ROCK inhibitor (Y-27632), a ERK1/2 inhibitor (PD0325901) a GSK3β inhibitor (CHIR99021), and a HDAC inhibitor (VPA). 8C: a p38 inhibitor (SB202190,), a JNK inhibitor (SP600125), a protein kinase C inhibitor (Go6983), a ROCK inhibitor (Y-27632), a ERK1/2 inhibitor (PD0325901), a GSK3β inhibitor (CHIR99021), a HDAC inhibitor (VPA), and a BMPa AMPK/BMP inhibitor (Dorsomorphin)].

### DETAILED DESCRIPTION OF THE INVENTION

Unless defined otherwise, all technical and scientific terms used herein have the same meaning as commonly understood by a person skilled in the art to which this invention belongs.

### 1. Definitions

As used herein, the singular forms “a”, “an”, and “the” include plural referents unless the context clearly dictates otherwise. Thus, for example, reference to “a component” includes a plurality of such components and equivalents thereof known to those skilled in the art.

The term “comprise” or “comprising” is generally used in the sense of include/including which means permitting the presence of one or more features, ingredients or components. The term “comprise” or “comprising” encompasses the term “consists” or “consisting of.”

As used herein, “mesenchymal stromal/stem cells (MSCs)” can self-renew and are multipotent. The term “multipotency” herein refers to a stem cell that has the ability to differentiate into more than one cell types. Multipotent stem cells cannot give rise to any type of mature cells in the body; they are restricted to a limited range of cell types. For example, MSCs can differentiate into osteoblasts, adipocytes, chondrocytes, neurons, β islet cells, intestine cells. MSCs can be obtained from various sources, such as bone marrow (BMMSCs), adipose or dental tissues and then cultured for expansion.

As used herein, the term “induced mesenchymal stem cells (iMSCs)” refers to MSC-like cells (i.e. cells having MSC-like features) which are generated (or dedifferentiated/reprogrammed) from other cell types, like skin cells. iMSCs are multipotent e.g. capable of differentiating into specific cells e.g. osteoblasts, chondrocytes, and adipocytes. As used herein, “skin cells” means cells found in skin such as epithelial cells or fibroblasts.

As used herein, the term “dedifferentiation” refers to a process where more differentiated cells are reverted to more primitive cells.

As used herein, the term “reprogram” refers to a process that convers cells into different cell types with some different properties or biological functions. For example, cells that are terminal differentiated can be reprogrammed to a multipotent stem cells. In case skin cells convert to multipotent MSCs with chemicals, the resulting cells are called “chemically induced MSCs (iMSCs)”.

As used herein, the term “culture” refers to a group of cells incubated with a medium. The cells can be passaged. A cell culture can be primary culture which has not been passaged after being isolated from the animal tissue, or can be passaged multiple times (subculture one or more times).

As used herein, a “kinase inhibitor” refers to an agent that can downregulate, decrease or suppress the amount and/or activity of a kinase which may be achieved by, for example, binding directly to the kinase protein, denaturing or otherwise inactivating the kinase, or inhibiting the expression of the gene (e.g. transcription to mRNA, translation of a polypeptide and/or modification to a mature protein) encoding the kinase, or a mutant in the sequence that can block the kinase activity. In general, kinase inhibitors may be proteins, polypeptides, nucleic acids, small molecules, or other chemical moieties. Assays to identify a kinase inhibitor are available in this art, such as western blotting.

As used herein, an “enzyme inhibitor” refers to an agent that can downregulate, decrease or suppress the amount and/or activity of an enzyme which may be achieved by, for example, binding directly to the enzyme protein, denaturing or otherwise inactivating the enzyme, or inhibiting the expression of the gene (e.g. transcription to mRNA, translation of a polypeptide and/or modification to a mature protein) encoding the enzyme, or a mutant in the sequence that can block the enzyme activity. In general, enzyme inhibitors may be proteins, polypeptides, nucleic acids, small molecules, or other chemical moieties. Assays to identify an enzyme inhibitor are available in this art, such as western blotting or enzyme activity assay.

The term “small molecule” as used herein refers to organic or inorganic molecules either synthesized or found in nature, generally having a molecular weight less than 10,000 grams per mole, particularly less than 5,000 grams per mole, particularly less than 2,000 grams per mole, and particularly less than 1,000 grams per mole. In some embodiments, a small molecule refers to a non-polymeric, e.g. non-protein or nucleic acid based, chemical molecule.

The term “about” as used herein means plus or minus 10% of the numerical value of the number with which it is being used. Therefore, about 1% means in the range of 0.9% to 1.1%.

A kinase inhibitor as described herein includes, for example, a p38 inhibitor, a c-jun N terminal kinase (JNK) inhibitor, a Rho-associated protein kinase (ROCK) inhibitor, an extracellular regulated kinase (ERK) inhibitor, an AMP-activated protein kinase (AMPK) inhibitor, a Src tyrosine kinase inhibitor, an anaplastic lymphoma kinase (ALK) inhibitor, a phosphoinositide 3-kinase inhibitor (PI3K) inhibitor, a tumor growth factor beta (TGFβ) inhibitor (e.g. RepSox), a molecular target of rapamycin (mTOR) inhibitor, and any combination thereof. These kinase inhibitors can be commercially available in this art. Other enzyme inhibitors as used herein include a histone deacetylase (HDAC) inhibitor, a G9 methyltransferase inhibitor, and a DOT1L inhibitor, for example.

Examples of PKC inhibitors as described herein include, but are not limited to, Go6976, Go66850, Go6983, rottlerin), bisindolylmaleimide II, C-1, calphostin C, melittin, GF 109203X, dihydrosphingosine, chelerythrine, chloride, CGP 53353, CID 2858522, Dihydrosphingosine, GF 109203X, Go 6976, Go 6983, [Ala107]-MBP (104-118), Ala¹¹³]-MBP (104-118), (±)-Palmitoylcarnitine chloride, PKC (19-36) (pseudosubstrate peptide; inhibitor of PKC), PKC 412, PKC pseudo substrate, Ro 32-0432 hydrochloride, rottlerin, D-erythro-sphingosine (synthetic), ZIP, and others.

Examples of GSK3β inhibitors as described herein include, but are not limited to, CHIR 99021, CHIR 99021 trihydrochloride, BIO, BIO-acetoxime, 3F8, AR-A 014418, TWS 119, TCS 2002, SB 216763, SB 415286, L803,and others.

Examples of p38 inhibitors as described herein include, but are not limited to, SB202190, SB 242235, EO 1428, Org 48762-0, SD 169, SB 203580, SB 202190, SB 239063, SB 220025, RWJ 67657,VX 745, VX 702, SD-282, SCIO 469, PH-797804, and others.

Examples of JNK inhibitors as described herein include, but are not limited to, SP600125, TCS JNK 5a, TCS JNK 6o, AEG 3482, BI 78D3, CEP 1347,IQ 1S, IQ3, and others.

Examples of ROCK inhibitors as described herein include, but are not limited to, Y-27632, AS 1892802, GSK 269962, GSK 429286, H 1152 dihydrochloride, HA 1100 hydrochloride, OXA 06 dihydrochloride, RKI 1447 dihydrochloride, SB 772077B dihydrochloride, etc.

Examples of ERK inhibitors as described herein include, but are not limited to, PD 98059 (a highly selective inhibitor of MEK1 and MEK2), selumetinib (also known as AZD6244), ARRY-438162, PD198306, PD0325901, AZD8330, PD184352 (also called CI-1040), PD184161, SL327, U0126, GW5074, BAY 43-9006, Ro 09-2210, FR 1 80204 PKI-ERK-005, ARRY-704, GSK 120212, RDEA1 19, XL518, CAY10561, and others.

Examples of AMPK inhibitors as described herein include, but are not limited to, dorsomorphin (6-[4-(2-Piperidin-1-yl-ethoxy)-phenyl]-3-pyridin-4-yl-pyrrazolo[1,5,-α]pyrimidine), BML-275, and others.

Examples of BMP inhibitors as described herein include, but are not limited to, dorsomorphin (6-[4-(2-Piperidin-1-yl-ethoxy)-phenyl]-3-pyridin-4-yl-pyrrazolo[1,5,-α]pyrimidine), and others.

Examples of Src tyrosine kinase inhibitors as described herein include, but are not limited to, PP1 (4-amino-5-(4-methylphenyl)-7-(t-butyl)pyrazolo[3,4-d]-pyrimidine), PP2, dasatinib, A 419259 trihydrochloride, AZM 475271, Bosutinib, Herbimycin A, KB SRC 4, LCB 03-0110 dihydrochloride, MNS, 1-Naphthyl PP1, Piceatannol, WH-4-023, Src I1, and others.

Examples of ALK inhibitors include, but are not limited to, SB431542, A 83-01, SB 505124, and others.

Examples of PI3K inhibitors include, but are not limited to, LY294002, A66, AS 252424, AS 605240, AZD 6482, BAG 956, CZC 24832, ETP 45658, GSK 1059615, KU 0060648, LY 294002 hydrochloride, 3-Methyladenine, PF 04691502, PF 05212384, PI 103 hydrochloride, PI 828, PP 121, Quercetin, TG 100713, TGX 221, Wortmannin, and others.

Examples of DOT1L methyltransferase inhibitors include, but are not limited to, SGC 0946, EPZ 004777, and others.

Examples of GLP and G9a histone lysine methyltransferase inhibitors include, but are not limited to, BIX 01294, A 366, UNC 0224, UNC 0638, UNC 0642, UNC 0646, and others.

Examples of mTOR inhibitors include, but are not limited to, rapamycin (sirolimus), temsirolimus, everolimus, the rapamycin prodrug AP-23573, AP-23481, the like, and combinations thereof.

As used herein, a cyclic adenosine monophosphate (cAMP) activator refers to an agent that increases intracellular levels of cAMP as compared to the background physiological intracellular level when the agent is absent. Examples of cAMP activators include, but are not limited to, forskolin, rolipram, NKH477, PACAP1-27, PACAP1-38 and others.

As used herein, a histone deacetylase (HDAC) inhibitor refers to an agent that downregulates, decreases or suppresses the amount and/or activity of histone deacetylase to remove acetyl groups from lysine residues on histones. Examples of HDAC inhibitor include, but are not limited to, valproic acid (VPA, 2-Propylpentanoic acid), Apicidin, CI 994, FK 228, LMK 235, M 344, MC 1568, MC 1742, MI 192, NCH 51, NSC 3852, PCI 34051, Sodium 4-Phenylbutyrate, Pyroxamide, SAHA, SBHA, Scriptaid, Sodium butyrate, TC-H 106, TCS HDAC6 20b, Trichostatin A, Tubacin, OF 010, and others.

As used herein, an antioxidant refers to an agent capable of slowing or preventing the oxidation of other molecules. Examples of antioxidants include, but are not limited to, vitamin E, beta-carotene, ascorbic acid (vitamin C), and a thiol-comprising compound (i.e., compounds comprising the functional group composed of a sulfur and a hydrogen atom, referred to as —SH), such as glutathione and the glutathione precursor N-acetylcysteine (NAC).

As used herein, a tumor growth factor beta (TGFβ) inhibitor refers to an agent that downregulates, decreases or suppresses the amount and/or activity of TGFβ, which may be achieved by, for example, binding to the TGFβ or inhibiting induction of TGFβ signaling through interaction with a factor in the TGFβ pathway. Examples of TGFβ inhibitors include, but are not limited to, RepSox, A 83-01, D 4476, GW 788388, LY 364947, R 268712, SB 431542, SB 505124, SB 525334, SD 208, and others.

As used herein, an auxiliary agent to enhance the efficacy of dedifferentiation/reprogramming from skin cells e.g. fibroblasts to iMSCs refers to an agent that can increase or improve the efficacy of dedifferentiation/reprogramming from skin cells to iMSCs when the skin cells are cultured with a PKC inhibitor and/or a GSK3β inhibitor in combination with such agent, as compared with that when the skin cells are cultured with a PKC inhibitor and/or a GSK3β inhibitor in the absence of the agent.

As used herein, the term “multipotency” herein refers to a stem cell that has the ability to differentiate into more than one cell types. A multipotent stem cell can become at least one or two certain cell type. For example, MSCs can differentiate into osteoblasts, adipocytes, and chondrocytes.

As used herein, the term “an isolated or purified population of cells” or “isolated or purified cells” refer to a preparation of cells that have been separated from other cellular components or other cells with which the cells are associated. For example, an isolated cell may have been removed from its native environment or group of cells, or may result from propagation of a cell that has been removed from a group of cells. When cells are described as “isolated” or “purified,” it should be understood as not absolutely isolated or purified, but relatively isolated or purified. For example, a preparation comprising isolated cells may comprise the cells in an amount of 0.5% or more, 10% or more, 20% or more, 30% or more, 40% or more, 50% or more, 60% or more, 70% or more, 80% or more, 90% or more, or 100% of the total cell number in the preparation. In some particular embodiments, a preparation comprising isolated cells may comprise the cells in an amount of 50% or more, 60% or more, 70% or more, 80% or more, 90% or more, or 100% of the total cell number in the preparation.

As used here, the term “subject” as used herein includes human and non-human animals such as companion animals (such as dogs, cats and the like), farm animals (such as cows, sheep, pigs, horses and the like), or laboratory animals (such as rats, mice, guinea pigs and the like).

As used herein, the term “treating” when relating to therapeutically treating refers to the application or administration of a composition including one or more active agents to a subject afflicted with a disorder, a symptom or conditions of the disorder, or a progression of the disorder, with the purpose to cure, heal, alleviate, relieve, alter, remedy, ameliorate, improve, or affect the disorder, the symptoms or conditions of the disorder, the disabilities induced by the disorder, or the progression of the disorder. On the other hand, the term “treating” can refer to an application or a process irrelevant to therapeutically treating a disease, such as applying one or more ingredients or agents to contact cells so as to change their fate e.g. reverting to other cell types.

As used herein, the term “therapeutically effective amount” used herein refers to the amount of an active ingredient to confer a therapeutic effect in a treated subject. The therapeutically effective amount may change depending on various reasons, such as administration route and frequency, body weight and species of the individual receiving said pharmaceutical, and purpose of administration. As used herein, the term “effective amount” when referring to an application or a process irrelevant to therapeutically treating a disease can refer to the amount of an ingredient or agent to be applied to achieve the intended purpose e.g. the amount of an ingredient or agent to be applied to contact cells e.g. fibroblasts for the purpose of dedifferentiation.

### 2. Use of Chemical Agents to Generate iMSCs

The present invention is based on an unexpected finding that skin cells e.g. fibroblasts can be dedifferentiated/reprogrammed into induced mesenchymal stem cells (iMSCs) by incubation with a PKC inhibitor and/or a GSK3β inhibitor, without gene modulation.

According to the present invention, skin cells can be cultured in a medium containing a PKC inhibitor and/or a GSK3β inhibitor in amount(s) effective in inducing dedifferentiation/reprogramming such that the skin cells are converted to iMSCs.

Culture media suitable for culturing skin cells according to the present invention are available in this art, such as DMEM, MEM, or IMEM medium. The culture can be carried out at in a normal condition, for example, 37° C. under 1-5% CO₂. Specifically, the culture medium can be serum free.

In some embodiments, the culture medium for conversion (conversion medium) contains knockout DMEM, AIbuMAX I, N2 supplement, nonessential amino acids (NEAA).

In some embodiments, the culture is carried out for at least 1 day or more (e.g. 2 days, 3 days, 4 days, 5 days, 6 days, 7 days, 8 days, 9 days, 10 days or more), whereby a proportion of the skin cells are converted into iMSCs.

In some embodiments, an auxiliary agent can be added to the culture medium to enhance the efficacy of dedifferentiation/reprogramming from fibroblasts to iMSCs. The auxiliary agent as used herein is selected from the group consisting of a p38 inhibitor, a JNK inhibitor, a ROCK inhibitor, an ERK inhibitor, a AMPK inhibitor, a Src tyrosine kinase inhibitor, an ALK inhibitor, a PI3K inhibitor, a cAMP activator, a HDAC inhibitor, an antioxidant, a TGFβ inhibitor, a mTOR inhibitor, G9a methyltransferase inhibitor, a DOTIL inhibitor, and any combination thereof. The auxiliary agent as used herein are added to the medium in an amount effective in enhance the efficacy of dedifferentiation/reprogramming from skin cells to iMSCs.

In some embodiments, the culture medium where the skin cells e.g. fibroblasts are cultured and reprogrammed into iMSCs comprises a combination of a PKC inhibitor and/or a GSK3β inhibitor and/or one or more auxiliary agents. Examples of such combination are as follows:

(1) a combination of a PKC inhibitor and a ROCK inhibitor;

(2) a combination of a PKC inhibitor, a ALK inhibitor and a ROCK inhibitor;

(3) a combination of a PKC inhibitor and a Src family tyrosine kinase inhibitor;

(4) a combination of a PKC inhibitor and a GSK3β inhibitor;

(5) a combination of a PKC inhibitor and a HDAC inhibitor;

(6) a combination of a PKC inhibitor, a HDAC inhibitor and a Src tyrosine kinase inhibitor;

(7) a combination of a PKC inhibitor, a HDAC inhibitor and a target of rapamycin (mTOR) inhibitor;

(8) a combination of a PKC inhibitor and a cAMP activator;

(9) a combination of a PKC inhibitor, a HDAC inhibitor and a G9a methyltransferase inhibitor;

(10) a combination of a PKC inhibitor, a HDAC inhibitor and a DOTIL inhibitor;

(11) a combination of a PKC inhibitor, a HDAC inhibitor, a JNK inhibitor and a p38 inhibitor;

(12) a combination of a PKC inhibitor, a GSK3β inhibitor, a INK inhibitor, a p38 inhibitor, a ROCK inhibitor and a ERK inhibitor;

(13) a combination of a PKC inhibitor, a HDAC inhibitor and a cAMP activator;

(14) a combination of a PKC inhibitor and a AMPK/BMP inhibitor;

(15) a combination of a PKC inhibitor, a GSK3β inhibitor and a HDAC inhibitor;

(16) a combination of a PKC inhibitor, a GSK3β inhibitor, a HDAC inhibitor, a ROCK inhibitor and an ERK inhibitor and;

(17) a combination of a PKC inhibitor, a HDAC inhibitor and a AMPK inhibitor /BMP inhibitor;

(18) a combination of a PKC inhibitor, a GSK3β inhibitor, a HDAC inhibitor and a AMPK/BMP inhibitor;

(19) a combination of a PKC inhibitor, a GSK3β inhibitor, a HDAC inhibitor, a JNK inhibitor, a p38 inhibitor, a ROCK inhibitor and a ERK inhibitor; and

(20) a combination of a PKC inhibitor, a GSK3β inhibitor, a HDAC inhibitor, a INK inhibitor, a p38 inhibitor, a ROCK inhibitor, a ERK inhibitor, and a AMPK inhibitor/BMP inhibitor.

In certain embodiments, the culture medium where the skin cells are cultured and reprogrammed into iMSCs comprises a combination of a PKC inhibitor with a GSK3β inhibitor, optionally with one or more auxiliary agents. Examples of such combination are as follows:

(4) a combination of a PKC inhibitor and a GSK3β inhibitor;

(12) a combination of a PKC inhibitor, a GSK3β inhibitor, a JNK inhibitor, a p38 inhibitor, a ROCK inhibitor and a ERK inhibitor;

(15) a combination of a PKC inhibitor, a GSK3β inhibitor and a HDAC inhibitor;

(16) a combination of a PKC inhibitor, a GSK3β inhibitor, a HDAC inhibitor, a ROCK inhibitor and an ERK inhibitor and;

(18) a combination of a PKC inhibitor, a GSK3β inhibitor, HDAC inhibitor and a AMPK/BMP inhibitor;

(19) a combination of a PKC inhibitor, a GSK3β inhibitor, a HDAC inhibitor, a JNK inhibitor, a p38 inhibitor, a ROCK inhibitor and a ERK inhibitor; and

(20) a combination of a PKC inhibitor, a GSK3β inhibitor, a HDAC inhibitor, a JNK inhibitor, a p38 inhibitor, a ROCK inhibitor, a ERK inhibitor, and a AMPK/BMP inhibitor.

In certain embodiments, the culture medium where the skin cells are cultured and reprogrammed into iMSCs comprises a combination of a PKC inhibitor with a HDAC inhibitor, optionally with a GSK3β inhibitor and/or one or more additional auxiliary agents. Examples of such combination are as follows:

(5) a combination of a PKC inhibitor and a HDAC inhibitor;

(6) a combination of a PKC inhibitor, a HDAC inhibitor and a Src tyrosine kinase inhibitor;

(7) a combination of a PKC inhibitor, a HDAC inhibitor and a target of rapamycin (mTOR) inhibitor;

(9) a combination of a PKC inhibitor, a HDAC inhibitor and a G9a methyltransferase inhibitor;

(10) a combination of a PKC inhibitor, a HDAC inhibitor and a DOT1L inhibitor;

(11) a combination of a PKC inhibitor, a HDAC inhibitor, a JNK inhibitor and a p38 inhibitor;

(13) a combination of a PKC inhibitor, a HDAC inhibitor and a cAMP activator;

(15) a combination of a PKC inhibitor, a GSK3β inhibitor and a HDAC inhibitor;

(16) a combination of a PKC inhibitor, a GSK3β inhibitor, a HDAC inhibitor, a ROCK inhibitor and an ERK inhibitor and;

(17) a combination of a PKC inhibitor, a HDAC inhibitor and a AMPK inhibitor;

(18) a combination of a PKC inhibitor, a GSK3β inhibitor, HDAC inhibitor and a AMPK/BMP inhibitor;

(19) a combination of a PKC inhibitor, a GSK3β inhibitor, a HDAC inhibitor, a JNK inhibitor, a p38 inhibitor, a ROCK inhibitor and a ERK inhibitor; and

(20) a combination of a PKC inhibitor, a GSK3β inhibitor, a HDAC inhibitor, a JNK inhibitor, a p38 inhibitor, a ROCK inhibitor, a ERK inhibitor, and a AMPK inhibitor BMP inhibitor.

In certain embodiments, the culture medium where the skin cells are cultured and reprogrammed into iMSCs comprises a combination of a PKC inhibitor with a GSK3β inhibitor and a HDAC inhibitor, optionally with one or more additional auxiliary agents. Examples of such combination are as follows:

(15) a combination of a PKC inhibitor, a GSK3β inhibitor and a HDAC inhibitor;

(16) a combination of a PKC inhibitor, a GSK3β inhibitor, a HDAC inhibitor, a ROCK inhibitor and an ERK inhibitor and;

(18) a combination of a PKC inhibitor, a GSK3β inhibitor, HDAC inhibitor and a AMPK/BMP inhibitor;

(19) a combination of a PKC inhibitor, a GSK3β inhibitor, a HDAC inhibitor, a JNK inhibitor, a p38 inhibitor, a ROCK inhibitor and a ERK inhibitor; and

(20) a combination of a PKC inhibitor, a GSK3β inhibitor, a HDAC inhibitor, a JNK inhibitor, a p38 inhibitor, a ROCK inhibitor, a ERK inhibitor, and a AMPK inhibitor BMP inhibitor.

In certain embodiments, the culture medium where the skin cells are cultured and reprogrammed into iMSCs comprises a combination of a PKC inhibitor with an AMPK/BMP inhibitor, optionally with a GSK3β inhibitor and/or one or more additional auxiliary agents. Examples of such combination are as follows:

(14) a combination of a PKC inhibitor and a AMPK/BMP inhibitor;

(17) a combination of a PKC inhibitor, a HDAC inhibitor and a AMPK/BMP inhibitor;

(18) a combination of a PKC inhibitor, a GSK3β inhibitor, HDAC inhibitor and a AMPK/BMP inhibitor;

(20) a combination of a PKC inhibitor, a GSK3β inhibitor, a HDAC inhibitor, a JNK inhibitor, a p38 inhibitor, a ROCK inhibitor, a ERK inhibitor, and a AMPK inhibitor/BMP inhibitor.

In certain embodiments, the culture medium where the skin cells are cultured and reprogrammed into iMSCs comprises a combination of a PKC inhibitor with a ROCK inhibitor, optionally with a GSK3β inhibitor and/or one or more additional auxiliary agents. Examples of such combination are as follows:

(1) a combination of a PKC inhibitor and a ROCK inhibitor;

(2) a combination of a PKC inhibitor, a ALK inhibitor and a ROCK inhibitor;

(12) a combination of a PKC inhibitor, a GSK3β inhibitor, a JNK inhibitor, a p38 inhibitor, a ROCK inhibitor and a ERK inhibitor;

(16) a combination of a PKC inhibitor, a GSK3β inhibitor, a HDAC inhibitor, a ROCK inhibitor and an ERK inhibitor and;

(18) a combination of a PKC inhibitor, a GSK3β inhibitor, HDAC inhibitor and a AMPK/BMP inhibitor;

(19) a combination of a PKC inhibitor, a GSK3β inhibitor, a HDAC inhibitor, a JNK inhibitor, a p38 inhibitor, a ROCK inhibitor and a ERK inhibitor; and

(20) a combination of a PKC inhibitor, a GSK3β inhibitor, a HDAC inhibitor, a JNK inhibitor, a p38 inhibitor, a ROCK inhibitor, a ERK inhibitor, and an AMPK inhibitor/BMP inhibitor.

In certain embodiments, the culture medium where the skin cells are cultured and reprogrammed into iMSCs comprises a combination of a PKC inhibitor with a GSK3β inhibitor and a ROCK inhibitor, further with a JNK inhibitor, a p38 inhibitor and a ERK inhibitor, optionally with one or more additional auxiliary agents. Examples of such combination are as follows:

(12) a combination of a PKC inhibitor, a GSK3β inhibitor, a JNK inhibitor, a p38 inhibitor, a ROCK inhibitor and a ERK inhibitor;

(19) a combination of a PKC inhibitor, a GSK3β inhibitor, a HDAC inhibitor, a JNK inhibitor, a p38 inhibitor, a ROCK inhibitor and a ERK inhibitor; and

(20) a combination of a PKC inhibitor, a GSK3β inhibitor, a HDAC inhibitor, a JNK inhibitor, a p38 inhibitor, a ROCK inhibitor, a ERK inhibitor, and a AMPK inhibitor/BMP inhibitor.

A number of PKC inhibitors and GSK3β inhibitor are available in this art. Table A illustrates some examples of the kinase inhibitors as used herein.

TABLE A

| Name/Source | Mechanism | Concentration | Structure |
|---|---|---|---|
| Go 6983, | PKC | 0.5-50 μM | 3-{1-[3-(Dimethylamino)propyl]-5- |
| Gö-6983 | inhibitor | | methoxy-1H-indol-3-yl}-4-(1H-indol-3- |
| (TOCRIS) | | | yl)-1H-pyrrole-2,5-dione |
| | | | 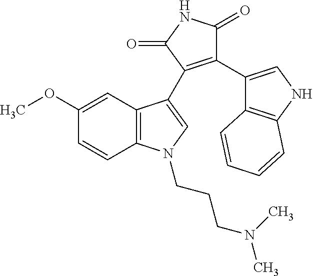 [SMILES: COc1ccc2c(c1)c(C1=C(c3c[nH]c4ccccc34)C(=O)NC1=O)cn2CCCN(C)C] |
| CHIR99021 | GSK3β | 0.3-30 μM | 6-[(2-{[4-(2,4-Dichlorophenyl)-5-(5- |
| (TOCRIS) | inhibitor | | methyl-1H-imidazol-2-yl)-2-pyrimidinyl] |
| | | | amino}ethyl)amino]nicotinonitrile |
| | | | 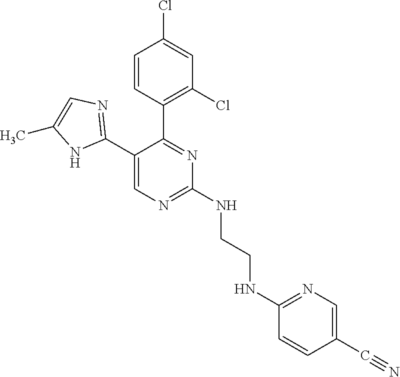 [SMILES: Cc1cnc(-c2cnc(NCCNc3ccc(C#N)cn3)nc2-c2ccc(Cl)cc2Cl)[nH]1] |
| LiCl | GSK-3β | 1-100 mM | Lithium chloride |
| (Sigma) | inhibitor, | | Cl⁻ Li⁺ |
| | LSD1 | | |
| | inhibitor | | |

Table B illustrates some examples of the auxiliary agent as used herein.

TABLE B

| Name/Source | Mechanism | Concentration | Structure |
|---|---|---|---|
| SB202190 (LC Laboratories) | p38 inhibitor | 1-100 μM | 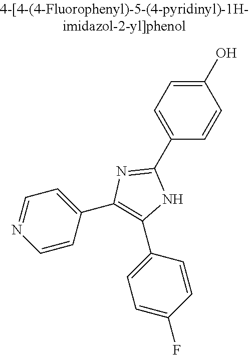 [SMILES: Oc1ccc(-c2nc(-c3ccncc3)c(-c3ccc(F)cc3)[nH]2)cc1] |
| SB203580 (LC Laboratories) | p38 inhibitor | 1-100 μM | 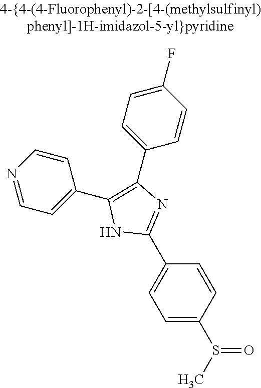 [SMILES: CS(=O)c1ccc(-c2nc(-c3ccc(F)cc3)c(-c3ccncc3)[nH]2)cc1] |
| SP600125 (LC Laboratories) | JNK inhibitor | 1-100 μM | 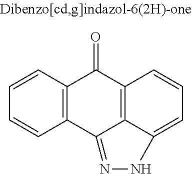 [SMILES: O=C1c2ccccc2-c2n[nH]c3cccc1c23] |
| Y-27632 (LC Laboratories) | Rho- associated protein kinase (ROCK) inhibitor | 0.5-50 μM | 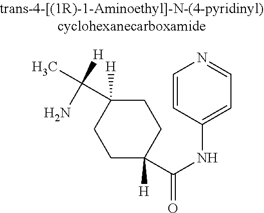 [SMILES: C[C@@H](N)[C@H]1CC[C@H](C(=O)Nc2ccncc2)CC1] |
| Thiazovivin (LC Laboratories) | Rho- associated protein kinase (ROCK) inhibitor | 0.1-50 μM | 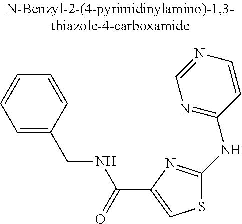 [SMILES: O=C(NCc1ccccc1)c1csc(Nc2ccncn2)n1] |
| PD0325901 | Erk | 0.1-50 μM | N-[(2S)-2,3-Dihydroxypropoxy]-3,4-difluoro- |
| (LC | inhibitor | | 2-[(2-fluoro-4-iodophenyl)amino]benz- |
| Laboratories) | | | amide |
| | | | 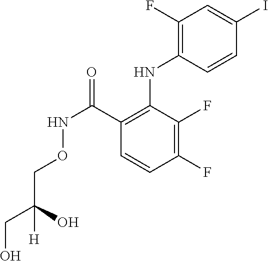 [SMILES: O=C(NOC[C@@H](O)CO)c1ccc(F)c(F)c1Nc1ccc(I)cc1F] |
| Dorsomorphin (LC laboratories) | BMP and AMPK inhibitor | 0.1-50 μM | 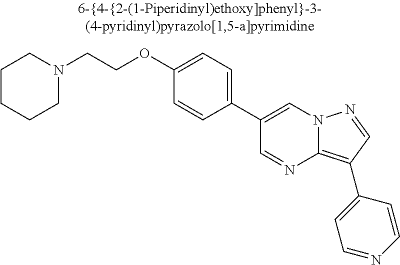 [SMILES: c1cc(-c2cnn3cc(-c4ccc(OCCN5CCCCC5)cc4)cnc23)ccn1] |
| PP1 (LC laboratories) | Replace Sox2, Src family tyrosine kinase inhibitor | 1-100 μM | 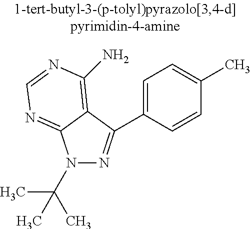 [SMILES: Cc1ccc(-c2nn(C(C)(C)C)c3ncnc(N)c23)cc1] |
| Dasatinib (LC laboratories) | Replace Sox2, Src family tyrosine kinase inhibitor | 1-100 μM | 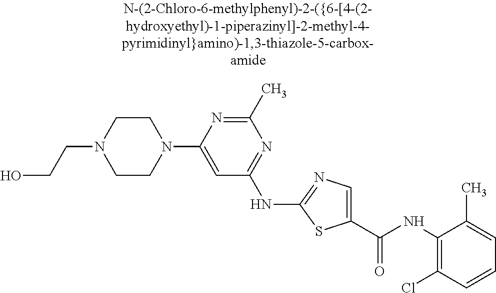 [SMILES: Cc1nc(Nc2ncc(C(=O)Nc3c(C)cccc3Cl)s2)cc(N2CCN(CCO)CC2)n1] |
| SB431542 (Sigma) | ALK4, ALK5, and ALK7 inhibitor | 1-100 μM | 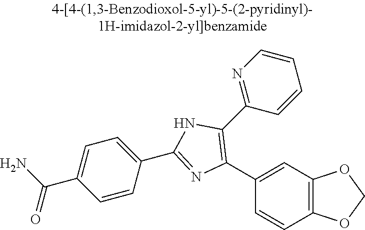 [SMILES: NC(=O)c1ccc(-c2nc(-c3ccc4c(c3)OCO4)c(-c3ccccn3)[nH]2)cc1] |
| LY294002 (LC laboratories) | Inhibitor of PI3K/AKT | 2-200 μM | 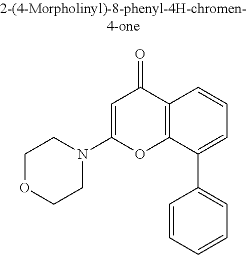 [SMILES: O=c1cc(N2CCOCC2)oc2c(-c3ccccc3)cccc12] |
| Rolipram (LC laboratories) | cAMP agonist | 1-100 μM | 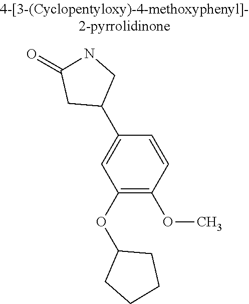 [SMILES: COc1ccc(C2CNC(=O)C2)cc1OC1CCCC1] |
| Forskolin, FSK | cAMP | 5-500 μM | (3R,4aR,5S,65,6aS,10S,10aR,10bS)-6,10,10b- |
| (TOCRIS) | activator | | Trihydroxy-3,4a,7,7,10a-pentamethyl-1- |
| | | | oxo-3-vinyldodecahydro-1H-benzo[f] |
| | | | chromen-5-yl acetate |
| | | | 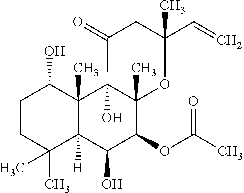 |
| Sodium butyrate (Sigma) | HDAC inhibitor | 0.1-10 mM | 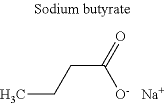 |
| Valproic acid (TOCRIS) | Histone deacetylase inhibitor | 0.2-20 mM | 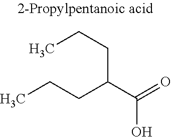 |
| N- Acetylcysteine, NAC (Sigma) | anti-oxidant small molecule | 0.2-20 mM | 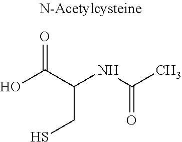 |
| Glutathione, GSH (Sigma) | Apoptosis regulator as a substrate of ROS scavenging enzymes | 0.2-20 mM | 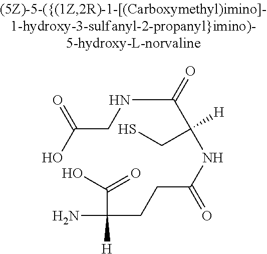 [SMILES: N[C@@H](CCC(=O)N[C@@H](CS)C(=O)NCC(=O)O)C(=O)O] |
| Vitamin C, VitC (Sigma) | Nanog enhancer, JAK/STAT activator | 5-500 ng/mL | 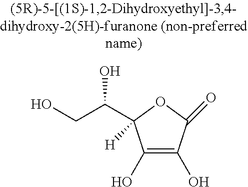 [SMILES: O=C1O[C@H]([C@@H](O)CO)C(O)=C1O] |
| RepSox | TGF-β | 1-100 μM | 2-[3-(6-Methyl-2-pyridinyl)-1H-pyrazol-4- |
| (TOCRIS) | inhibitor | | yl]-1,5-naphthyridine |
| | | | 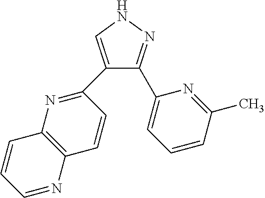 [SMILES: Cc1cccc(-c2n[nH]cc2-c2ccc3ncccc3n2)n1] |
| A8301 (Sigma) | TGF-β signaling inhibitor | 0.2-50 μM | 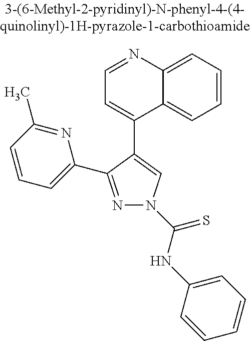 [SMILES: Cc1cccc(-c2nn(C(=S)Nc3ccccc3)cc2-c2ccnc3ccccc23)n1] |
| Rapamycin | mTOR | 0.03-3 nM | (1R,9S,12S,15R,16E,18R,19R,21R,23S,24E, |
| (TOCRIS) | inhibitor | | 26E,28E,30S,32S,35R)-1,18-Dihydroxy- |
| | | | 12-{(2R)-1-[(1S,3R,4R)-4-hydroxy-3-meth- |
| | | | oxycyclohexyl]-2-propanyl}-19,30-dimeth- |
| | | | oxy-15,17,21,23,29,35-hexamethyl-11,36- |
| | | | dioxa-4- |
| | | | azatricyclo[30.3.1.0~4,9~]hexatriaconta- |
| | | | 16,24,26,28-tetraene-2,3,10,14,20-pentone |
| | | | 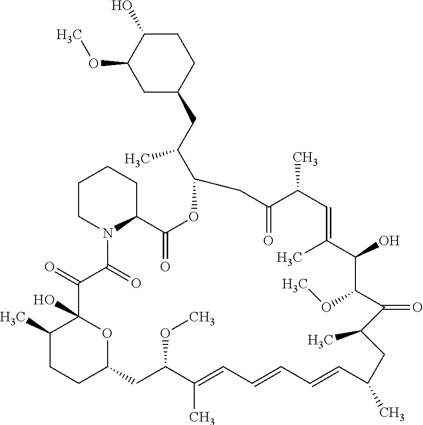 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](O)[C@H](OC)C2)CC(=O)[C@H](C)/C=C(\C)[C@@H](O)[C@@H](OC)C(=O)[C@H](C)C[C@H](C)/C=C/C=C/C=C/1C] |
| Tranylcypromine (TOCRIS) | H3K4 demethylation inhibitor | 1-100 μM | 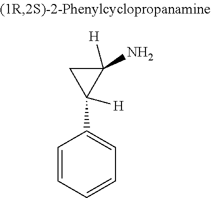 |
| 3- Deazaneplanocin A, DZNep (TOCRIS) | 5-adenosyl methionine- dependent methyltrans- ferase | 0.01-1 μM | 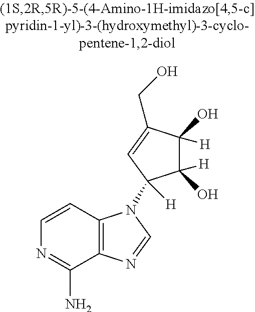 [SMILES: Nc1nccc2c1ncn2[C@@H]1C=C(CO)[C@@H](O)[C@H]1O] |
| TTNPB | potent | 0.1-50 μM | 4-[(1E)-2-(5,5,8,8-Tetramethyl-5,6,7,8-tetra- |
| (TOCRIS) | analog of | | hydro-2-naphthalenyl)-1-propen-1-yl] |
| | retinoic acid | | benzoic acid |
| | | | 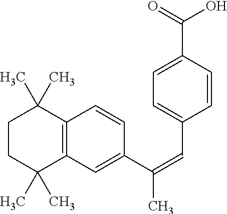 [SMILES: C/C(=C/c1ccc(C(=O)O)cc1)c1ccc2c(c1)C(C)(C)CCC2(C)C] |
| uercetin | HIF1α | 0.1-10 μM | 2-(3,4-Dihydroxyphenyl)-3,5,7-trihydroxy- |
| (TOCRIS) | activator | | 4H-chromen-4-one |
| | | | 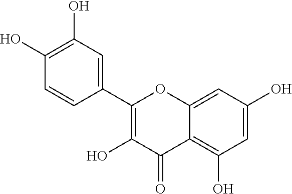 [SMILES: O=c1c(O)c(-c2ccc(O)c(O)c2)oc2cc(O)cc(O)c12] |
| CoCl₂ (Sigma) | HIF1α activator | 10-1000 μM | 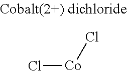 |
| ML228 | HIF1α | 1-100 μM | N-(4-Biphenylylmethyl)-6-phenyl-3-(2- |
| (TOCRIS) | activator | | pyridinyl)-1,2,4-triazin-5-amine |
| | | | 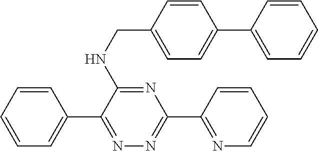 [SMILES: c1ccc(-c2ccc(CNc3nc(-c4ccccn4)nnc3-c3ccccc3)cc2)cc1] |
| 5-aza-CR, AZA | DMNT | 0.05-5 mM | 4-Amino-1-(beta-D-ribofuranosyl)-1,3,5-tri- |
| (Sigma) | inhibitor | | azin-2(1H)-one |
| | | | 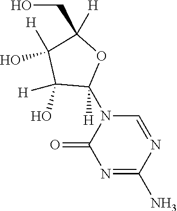 [SMILES: Nc1ncn([C@@H]2O[C@H](CO)[C@@H](O)[C@H]2O)c(=O)n1] |
| I-BET 151 | BET family | 0.2-50 μM | 7-(3,5-Dimethyl-1,2-oxazol-4-yl)-8-methoxy- |
| (TOCRIS) | bromodomain | | 1-[(1R)-1-(2-pyridinyl)ethyl]-1,3-dihydro- |
| | inhibitor | | 2H-imidazo[4,5-c]quinolin-2-one |
| | | | 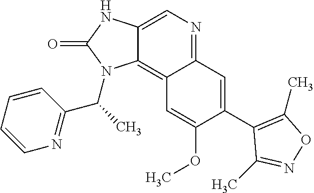 [SMILES: COc1cc2c(cc1-c1c(C)noc1C)ncc1[nH]c(=O)n([C@H](C)c3ccccn3)c12] |
| Fasudil (LC laboratories) | Rho kinase inhibitor | 0.02-50 μM | 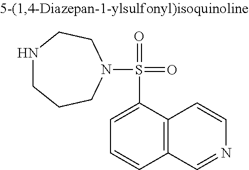 [SMILES: O=S(=O)(c1cccc2cnccc12)N1CCCNCC1] |
| SGC0946 | DOT1L | 0.5-50 μM | 5-Bromo-7-(5-deoxy-5-{isopropyl[3-({[4- |
| | inhibitor | | (2-methyl-2-propanyl)phenyl]carbamoyl} |
| | | | amino)propyl]amino}-β-D-ribofuranosyl)- |
| | | | 7H-pyrrolo[2,3-d]pyrimidin-4-amine |
| | | | 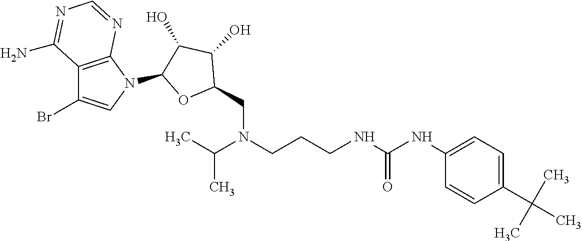 [SMILES: CC(C)N(CCCNC(=O)Nc1ccc(C(C)(C)C)cc1)C[C@H]1O[C@@H](n2cc(Br)c3c(N)ncnc32)[C@H](O)[C@@H]1O] |
| BIX01294 | G9a methyltrans- ferase inhibitor | 0.2-50 μM | 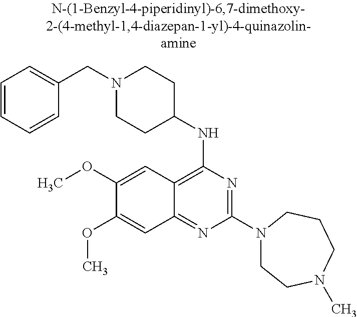 [SMILES: COc1cc2nc(N3CCCN(C)CC3)nc(NC3CCN(Cc4ccccc4)CC3)c2cc1OC] |

According to the present invention, about 1%, 3%, 5%, 10%, 15%, 20%, 25%, 30%, 35%, 40%, 45%, 50%, 55%, 60%, 65%, 70%, 75%, 80% or more of the skin cells in the culture are dedifferentiated/reprogrammed into iMSCs. In some certain embodiments, about 55% to 85%, of the skin cells in the culture are dedifferentiated into iMSCs.

### 3. Skin Cells for Use in Dedifferentiation/Reprogramming

Skin cells e.g. fibroblasts can be used herein to generate iMSCs in the present invention. Fibroblasts as used herein for dedifferentiation/reprogramming can be obtained from neonatal or adult donors.

Skin biopsies can be obtained from proper autologous or allogenic donors by skin puncture or circumcision and skin fibroblasts can be grown from the skin biopsies. In general, skin biopsies of about 4-mm can generate 15-20 million fibroblasts¹⁹. In some embodiments, commercial fibroblasts are available. Preferably, the fibroblasts for conversion into iMSCs as used herein are of mammalian origin, most preferably of human origin,

### 4. iMSCs

According to the present invention, the iMSCs as generated are of MSC-like features. Specifically, the iMSCs have MSC-like morphology, a small cell body with a few cell processes which is spindle cell like. More specifically, the iMSCs as generated can express typical MSC markers.

In some embodiments, the MSC marker is selected from the group consisting of stage-specific embryonic antigen (SSEA)-4 and podocalyxin-like protein (PODXL).

In some embodiments, the MSC marker is selected from the group consisting of CD105, CD73, CD44, CD90, a combination thereof.

In some embodiments, the iMSCs are negative for CD45, CD34, CD11b, and CD19.

In some embodiments, the iMSCs are SSEA-4⁺, PODXL⁺, CD105⁺, CD73⁺, CD44⁺, CD90⁺, CD45⁻, CD34⁻, CD11b⁻, CD19⁻.

In addition, the iMSCs as generated are multipotent which can differentiate into specific cell types, including osteoblasts (bone lineage), adipocytes (fat lineage), and chondrocytes (cartilage lineage). Further, the iMSCs as generated can also express immunomodulatory function. In certain examples, the iMSCs can inhibit acute ling injury as demonstrated in the animal model (see examples below). Moreover, the iMSCs can be expanded in culture and stored for later retrieval and use. In some embodiments, the iMSCs as generated in the present invention can be maintained and expanded for at least 3 passages, 4 passages, 5 passages, 6 passages, 7 passages or 8 passages or more.

Once a culture of iMSCs is established, the population of cells is mitotically expanded in vitro by passage to regular MSC medium during cell density controls under conditions conducive to cell proliferation, with or without tissue formation. Such culturing methods can include but are not limit to passaging the cells in culture medium with particular growth factors (e.g., IGF, EGF, FGF, VEGF, and/or other growth factors) or commercial medium Cultured cells can be transferred to regular MSC culture medium till cell density is reached. Thereby, proper passaging techniques can be used to reduce contact inhibition and maintain appropriate cell physiology.

Further, iMSCs can be cryopreserved for storage in a “freeze medium” containing 10-90% fetal bovine serum (FBS) and 10% dimethyl sulfoxide (DMSO), of about but not limit to 5×10⁵-1×10⁷ cells/ml. In some embodiments, the cells can be frozen with commercial medium. The cells are dispensed into plastic vials which are then transferred to a freezing chamber. Once vials containing the cells reached −80° C., they can be transferred to a liquid nitrogen for storage. Cryopreserved cells can be stored for a period of years.

After culture in some embodiment, to further enrich iMSCs, the cells can be sorted with one or more MSC markers. Cell sorting can be achieved by various techniques as known in the art. Examples of cell sorting techniques include fluorescence-activated cell sorting (FACS), immunoaffinity column separation or immunomagnetic separation (MACS) or any technique which is capable of obtaining enrichment of one certain cell type on the basis of physical characteristics (density) or structural characteristics (in particular specific antigens).

### 5. Applications of iMSCs

5.1 Differentiation into Specific Cells

Due to the multipotency, iMSCs, like natural MSCs, can be induced to differentiate into cells, such as fibroblasts, adipocytes, chondrocytes, osteoblasts, osteocytes, myoblasts, neurons, beta islet cells, hepatocytes, cardiomyocytes, neural stem cells, more typically fibroblasts, osteoblasts, osteocytes, chondroblasts, chondrocytes, adipocytes, and myocytes. Besides, iMSCs/MSCs can transdifferentiate into neural lineage cells or beta-cells of pancreas.

Differentiation of iMSCs to other cell types can be triggered by changing the culture conditions or by treating with specific exogenous growth factors. Methods for inducing differentiation of cells to a desired cell type are well known in the art.

Factors that can be used to induce iMSC differentiation include growth factors, enzyme, hormone, and other signaling molecule. For instance, beta-glycerophosphate (BGP), ascorbic acid and dexamethasone are crucial for osteogenesis; insulin, IBMS, indomethacin, and dexamethasone are crucial for adipogenesis; TGF-β and dexamethasone are crucial for chondrogenesis;

hydrocortisone and dexamethasone are crucial for myogenesis. The iMSCs also can be cultured with tissue committed cells to turn into a particular lineage.

5.2 Cell Therapies

Based on our findings, it is possible to generate autologous or allogenic iMSCs from accessible skin biopsy, which can be easily obtained in the clinic. This process does not require surgery or any other painful process. Given MSCs' ability to repair damaged tissues and immunomodulation functions, the diseases can be treated by iMSCs may include but not limited to heart diseases (e.g. peripheral arterial disease, ischemia, stroke, myocardial infraction), acute lung injury (ALI), graft-versus-host disease, Crohn's disease, type 1 diabetes mellitus, multiple sclerosis, neurological diseases, osteogenesis imperfecta, fibrosis, and inherited diseases such as Hurler's syndrome. Therapeutic uses of iMSCs include transplanting the iMSCs, stem cell populations, or progeny thereof into individuals to treat several different disease such as anti-inflammation (immunomodulatory capacity), cardiovascular disease, neurodegenerative disorders, tissue engineering and the like. Treatment may use the cells to construct new tissue (with or without biomaterials), according to any method known in the art. The cells, iMSCs or the progeny, may be injected or transplanted to the site of tissue damage so that they will produce new tissue in vivo. The iMSC-derived cells may be used in the clinic for tissue engineering and regenerative medicine. For example, iMSCs may be applicable in cartilage and bone regeneration for the treatments of arthritis, lower back pain (LBP), cartilage degeneration, bone fracture, or osteoporosis. In addition, since iMSCs can differentiate into fat and cartilage, iMSCs may also be applicable in plastic surgery such as autologous fat transplantation and cartilage grafting in nasal augmentation.

In a preferred embodiment, the patients can get the autologous skin cells to generate iMSCs for the treatment and do not need to take immunosuppressive drugs. In another embodiment, it will be easier to find donors willing to donate skin cells for iMSCs production while compared to the bone marrow aspiration for obtaining MSCs. If the human iMSCs are derived from a heterogeneous/allogenic source, concomitant immunosuppressive therapy is sometimes administered, for instance, administration of the immunosuppressive agent FK560 or cyclosporine. In some embodiment, the iMSCs or the progeny can be encapsulated in a membrane which can exchange but prevent cell and cell contact. Transplantation of microencapsulated is known in the art, e.g. Dixit et al., Cell Transplantation 1:275-79 (1992); and Balladur et al., Surgery, 117:198-94 (1995).

In addition, MSCs is well known for its immunomodulatory capacity as well as the function to regulate/suppress B cells, T cells, and NK cells in immune system. In particular, iMSCs as generated in the present invention are demonstrated to have the immunomodulatory capacity in mice model. As shown in the examples below, iMSCs as generated in the present invention are effective in reducing LPS-mediated acute lung injuries in animals.

### 6. Improvement of MSCs' Functional Characteristics by Chemical Treatment

The present invention is also based on an unexpected finding that MSCs after treatment with the chemical agent(s) as described herein exhibits enhanced MSC's functional characteristics.

In particular, the chemical treatment to improve MSC's functional characteristics as described herein include culturing MSCs in a culture medium which comprises a protein kinase C (PKC) inhibitor (e.g. GO6983) and/or a glycogen synthase kinase 3 beta (GSK3β) inhibitor (e.g. CHIR99021).

In some embodiments, the culture medium can further comprise an auxiliary agent as described herein, including but are not limited to a p38 inhibitor (e.g. SB202190, SB203580), a c-jun N terminal kinase (JNK) inhibitor (e.g. SP600125), a Rho-associated protein kinase (ROCK) inhibitor (e.g. Y-27632), an extracellular regulated kinase (ERK) inhibitor (e.g. PD325901), an AMP-activated protein kinase (AMPK) inhibitor (e.g. Dorsomorphin), a Src tyrosine kinase inhibitor (e.g. PP1, Dasatinib), an anaplastic lymphoma kinase (ALK) inhibitor (e.g. SB431542), a phosphoinositide 3-kinase inhibitor (PI3K) inhibitor (e.g. LY294002), a cyclic adenosine monophosphate (cAMP) activator (e.g. Froskolin, Rolipram), a histone deacetylase (HDAC) inhibitor (e.g. VPA), an antioxidant (e.g. NAC, GSH, etc.), a antioxidant (e.g. vitamin C), a tumor growth factor beta (TGFβ) inhibitor (e.g. RepSox), a molecular target of rapamycin (mTOR) inhibitor (e.g. Rapamycin), a G9a methyltransferase inhibitor (e.g. BIOX01294), a DOTIL inhibitor (e.g. SGC0946) and any combination thereof.

In one embodiment, the chemical treatment include merely a protein kinase C (PKC) inhibitor (e.g. GO6983).

In some embodiments, the chemical treatment include a combination of a p38 inhibitor (e.g. SB202190), a JNK inhibitor (e.g. SP600125), a protein kinase C inhibitor (e.g. Go6983), a ROCK inhibitor (e.g. Y-27632), a ERK1/2 inhibitor (e.g. PD0325901) and a GSK3β inhibitor (e.g. CHIR99021), optionally further comprising a HDAC inhibitor (VPA) and/or a BMPa AMPK/BMP inhibitor (Dorsomorphin).

Specifically, the functional characteristics, such as the activities in expansion, clonogenicity and/or differentiation (multipotency) of MSCs can be improved by the chemical treatment as described herein. The improvement of MSCs' functional characteristics can be determined by methods known in the art e.g. based on increase of expression of representative MSC markers, e.g. SSEA4+ and PODXL+, cell culture observation of clonogenicity and differentiation activity assays.

In general, the present invention provides a new technology to generate iMSCs and improving MSCs' functional characteristics including the features as follows:

(1) an easy and simple in vitro process to generate iMSCs from skin cells e.g. fibroblasts;

(2) the source of skin cells is easily accessible and there is no need to carry out surgery or other significantly painful process to obtain the skin cells (it is easier to find donors willing to donate skin cells for allogenic iMSC production while compared to the bone marrow aspiration or liposuction for obtaining MSCs);

(3) it is possible to generate autologous or allogenic iMSCs from accessible skin biopsies and then patients use autologous iMSCs do not need to take immunosuppressive drugs has better chance for long term engraftment, and increase the safety;

(4) merely chemical cocktail is required (no gene modification) to reprogram/de-differentiate skin cells into iMSCs (no prior technology can generate MSCs from fibroblasts with chemical agents and with chemical agents good for clinical application);

(5) iMSCs can be generated with a few chemical agents (with or without growth factors) very soon (within 6 days);

(6) iMSCs conversion rate from skin cells is high; the efficiency can reach about 80% in a preferred embodiment;

(7) both neonatal fibroblasts and adult skin fibroblasts can be converted into functional iMSCs (of note, it is hard to isolate functional and expandable MSCs from elderly patients as known in the art);

(8) iMSCs, like MSCs, are expandable for at least 8 passages;

(9) the components of cocktails for generating iMSCs are all well-defined and do not contain animal serum, which is suitable for clinical applications and has high reproducibility;

(10) iMSCs, like MSCs, are multipotent and can differentiate into multiple functional cell types;

(11) iMSCs, like MSCs, have immunomodulation functions and can treat diseases in the animal models;

(12) iMSCs as generated share the same molecular signatures with MSCs; and

(13) the reprogramming process only requires chemical agents (with or without growth factors); no retrovirus/lentivirus/plasmid to change the genetic information is required for the dedifferentiation/reprogramming process, which avoids insertional mutagenesis or other biosafety concerns.

(14) the processing can increase the population of MSC highly expressing SSEA-4 and PODXL. SSEA-4⁺ and PODXL⁺ cells are MSCs that harbors better expansion ability, clonogenicity, and differentiation ability. Functional MSCs are hard to isolate from elderly or some donors. This method may help to turn the cells cannot expand or differentiate well into functional MSCs.

The present invention is further illustrated by the following examples, which are provided for the purpose of demonstration rather than limitation. Those of skill in the art should, in light of the present disclosure, appreciate that many changes can be made in the specific embodiments which are disclosed and still obtain a like or similar result without departing from the spirit and scope of the invention.

### Examples

1. Material and Methods

1.1 Reagents

Culture media e.g. knockout Dulbecco's Modified Eagle's medium (DMEM) were purchased from Invitrogen (Carlsbad, Calif., USA). Chemicals e.g.

kinase inhibitors were purchased from LC Laboratories (Woburn, Mass., USA), TOCRIS (Bristol, UK), Sigma Aldrich (St. Louis, Mich., USA), or other company. Recombinant proteins e.g. recombinant growth factors were purchased from Peprotech (Rocky Hill, N.J., USA), R&D (Minneapolis, Minn., USA), or others.

1.2 Cell Culture

All experiments with primary human cells are approved by the Institutional Review Board (Taipei, Taiwan). Human primary neonatal foreskin fibroblasts (CRL2097) were purchased from American Type Culture Collection (ATCC) (Manassas, Va., USA) and cultured in Dulbecco's Modified Eagle Media-high glucose (DMEM-HG) medium with 10% fetal bovine serum (FBS) (HyClone, Logan, Utah, USA). Primary adult skin fibroblasts were derived from a 42 or 56-year-old female (LONZA, Basel, Switzerland) and cultured with DMEM-HG with 10% FBS. Human primary bone marrow mesenchymal stem cells (BMMSCs) were cultured with Dulbecco's Modified Eagle Media-low glucose (DMEM-LG) medium containing 10% FBS. All cells were cultured at 37° C. under 5% CO₂.

1.3 Generation of iMSCs

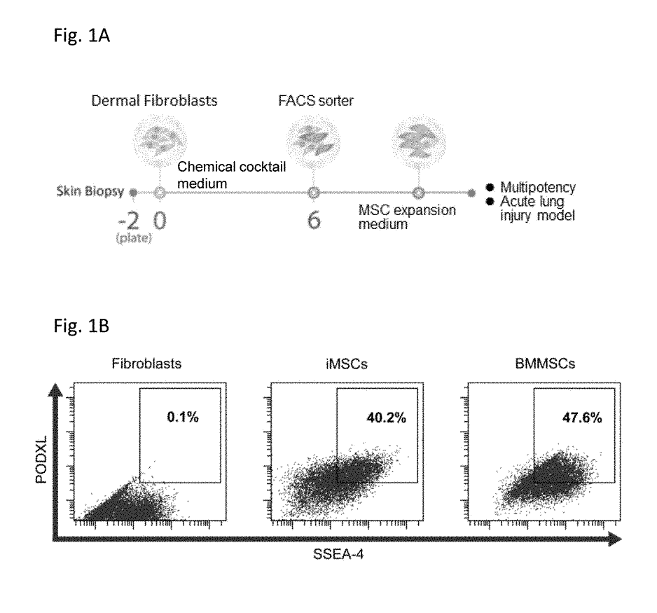

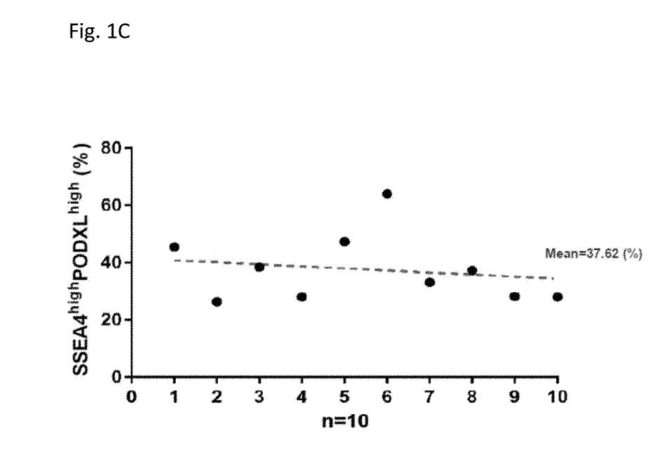

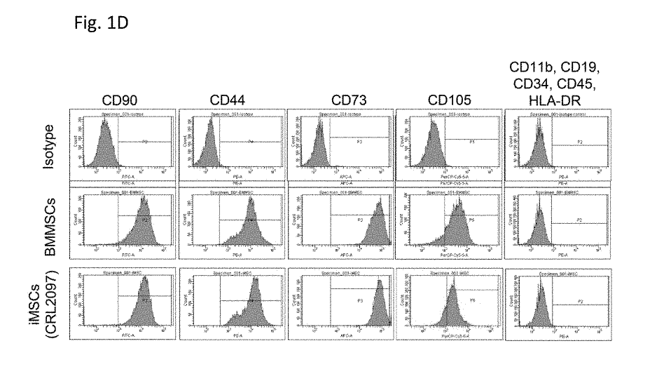

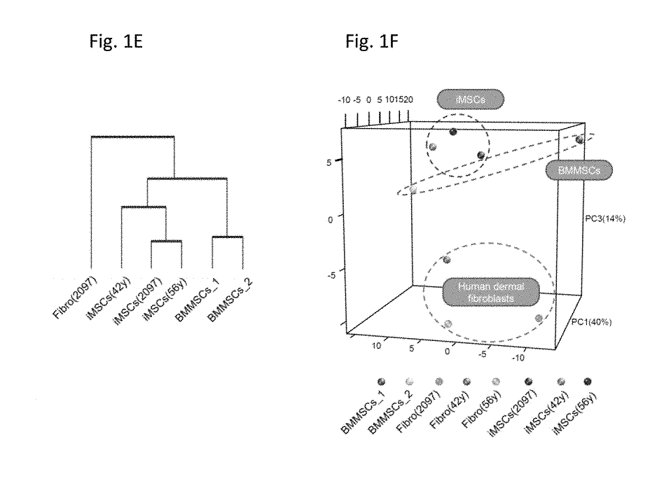

For iMSC dedifferentiation from human fibroblasts, primary fibroblasts were cultured in DMEM-HG for 2 days. The culture medium was then replaced with medium containing different chemical(s), with or without growth factor(s), for 3 to 21 days (FIG. 1A). Cells were cultured at 37° C. under 5% CO₂. The culture medium for conversion contains knockout DMEM, AlbuMAX I, N2 supplement, nonessential amino acids (NEAA), which did not contain any serum or undefined components.

1.4 Flow Cytometry and Cell Sorting

For cell surface marker analysis, BMMSCs, iMSCs, and fibroblasts were incubated with FITC-conjugated anti-human SSEA-4 (clone MC-813-70; eBiosciences, San Diego, Calif., USA) and PE-conjugated anti-human PODXL (clone B34D1.3; eBiosciences) antibodies. Cells were then analyzed by FACSCanto (Becton Dickinson, Franklin Lakes, N.J., USA). SSEA-4 and PODXL double positive cells were isolated using a cell sorter (FACS Aria II, BD Biosciences).

1.5 Microarray Analysis.

RNA was purified using an RNeasy kit (Qiagen, Hilden, Germany). Samples were prepared for array hybridization using the human Affymetrix 3′ IVT Express kit with GeneChip® Human Genome U133A 2.0 chips following the manufacturer's protocol (Affymetrix, Santa Clara, Calif., USA). The array results were analyzed with Gene Spring GX 12.6 software (Agilent Technologies, Santa Clara, Calif., USA). Microarray profiling of BMMSC_2 was described previously in Pubmed Geo data base (GSM1533333), and all other array data were uploaded to the Pubmed Geo data base (GSE72693). Genes with 4-fold or greater expression in iMSCs as compared to fibroblasts were used in this analysis. Log-transformed expression data were hierarchically clustered by samples and probes in GeneSpring.

1.6 Immunofluorescence Assay

Cells were fixed with 4% paraformaldehyde (PFA) for 15 min, permeabilized in 0.3% Triton X-100 for 10 min, and then blocked in 2% BSA in PBS for 1 hour. Cells were incubated in FITC-conjugated anti-human SSEA-4 (eBioscience) or PE-conjugated anti-human PODXL (eBioscience) for 1 hour at room temperature, and then washed 3 times with PBS. The nuclei of cells were counter stained with 4′,6-diamidino-2-phenylindole (DAPI) and fluorescence images were acquired using a LAS-4000 image system (Fujifilm, Tokyo, Japan). Finally, the brightness and contrast of whole images were adjusted linearly using Multi Gauge version 3.0 (Fujifilm).

1.7 Osteogenic Differentiation

Human primary BMMSCs and iMSCs were cultured in DMEM-LG medium plus 10% FBS. Fibroblasts were cultured in DMEM-HG plus 10% FBS. To induce differentiation, cells (1×10⁴ cells/cm²) were cultured with osteogenic-induction medium (90% DMEM-HG, 10% FBS, 0.1 μM dexamethasone, 10 mM beta-glycerophosphate, 0.05 mM L-ascorbic acid phosphate). Media were replaced twice per week during differentiation.

1.8 Alkaline Phosphatase Activity Assay

After 10 days of osteogenic differentiation, cells were fixed with 4% PFA in PBS for 3 min. Alkaline phosphatase (ALP) staining was performed using alkaline phosphatase kits in accordance with the manufacturer's instruction (Sigma). For quantification of ALP activity, the cells were washed twice with PBS, and incubated with ALP substrate p-nitrophenyl phosphate (pNPP) at room temperature for 5-20 minutes. Absorbance at an optical density (O.D.) of 405 nm was measured.

1.9 Alizarin Red S Staining

After 21 days of osteogenic differentiation, cells were fixed with ice-cold 70% ethanol at −20° C. for 1 hour and then washed with PBS. The cells were then stained with 40 mM Alizarin Red S (ARS) (pH 4.2) for 10 minutes, and subsequently washed five times with ddH₂O before being air dried. For quantification, cells were incubated with 1 mL cetylpyridinium chloride buffer for 1 h to extract ARS, and the O.D. at 550 nm was then recorded.

1.10 Adipogenic Differentiation

For adipogenic induction, cells were cultured in adipogenic induction medium (Biological industry, Kibbutz Beit-Haemek, Israel), which was replaced twice per week during the 21-day differentiation period.

1.11 Oil Red O Staining

Cells were fixed with 4% PFA for 1 hour, washed with 60% isopropanol, and then air dried. The lipid vesicles were stained with oil red O staining medium (30 ml of 0.5% oil red solution in 2-propanol, diluted with 20 ml of water) for 10 mins, and then washed with distilled water. For quantification, Oil Red O was extracted with isopropanol, and the absorbance at an O.D. of 530 nm was measured.

1.12 Chondrogenic Differentiation

For chondrogenic differentiation, BMMSCs, iMSCs, and fibroblasts (2.5×10⁵ cells) in separate 15 mL tubes were centrifuged at 500 g for 10 min; the pelleted cells were then incubated with chondrogenic induction medium (Biological industry). The cells formed a spherical aggregate after overnight incubation. The cells were continuously induced for 21 days, and paraffin sections were taken to analyze the samples. After deparaffinization, the slides were stained with Hematoxylin-eosin or Alcian blue solution.

1.13 Alcian Blue Staining

Pelleted cells were embedded in paraffin. After sectioning, slides were deparaffinized with xylene and hydrated with distilled water. After incubation of slides in 3% acetic acid for 3 min, the slides were stained with 1% Alcian Blue solution (in 3% acetic acid, pH 2.5) for 30-45 min. The slides were then washed with water for 2 min, dehydrated with xylene, and mounted with mounting solution (Thermo Fisher Scientific, Waltham, Mass., USA).

1.14 Endotoxin-Induced Acute Lung Injury in Mice

All animal experimental procedures were approved by the Animal Ethics Committee of Academia Sinica (Taipei, Taiwan). BALB/c female mice (6-8 weeks, National Laboratory Animal Center, Taipei, Taiwan) were first anesthetized with Tiletamine/Zolazepan (25 mg/kg) and xylazine (10 mg/kg) via the intraperitoneal route. Acute lung injury (ALI) was then induced by the intratracheal (i.t.) instillation of 40 mg/kg lipopolysaccharides (LPS) purified from E. coli 055:B5 (Sigma-Aldrich) or 100 μl PBS. At four hours after LPS treatment, mice were anesthetized again and then randomly divided into four groups: (1) PBS, (2) human fibroblasts (10⁶ cells in 100 μl PBS), (3) iMSCs (10⁶ cells in 100 μl PBS), and (4) human BMMSCs (10⁶ cells in 100 μl PBS). The survival of mice was followed for 48 h. Survival rate of each group was observed every 6 hours. For the histology analysis and lung injury analysis, the samples were collected before or at 48 hours post injection

1.15 Improvement of Multipotency of MSCs

BBMSCs were cultured in conversion medium for 6 days, without chemical treatment (control) or with treatment of chemical cocktails (6C+3GF), including six chemical kinase(6C) inhibitors i.e. a p38 inhibitor (5B202190), a JNK inhibitor (SP600125), a protein kinase C inhibitor (Go6983), a ROCK inhibitor (Y-27632), a ERK1/2 inhibitor (PD0325901) and a GSK3β inhibitor (CHIR99021) and three growth factors i.e. a leukemia inhibitory factor (LIF), a basic fibroblast growth factor (bFGF) and a transforming growth factor-β (TGF-β). In other conditions, MSCs were cultured with chemicals cocktails (six, seven or eight chemicals) without the growth factors. The percentages of the MSC markers, SSEA-4 and PODXL, were determined by flow cytometer. Six chemicals (6C) includes a p38 inhibitor (SB202190), a JNK inhibitor (SP600125), a protein kinase C inhibitor (Go6983), a ROCK inhibitor (Y-27632), a ERK1/2 inhibitor (PD0325901) and a GSK3β inhibitor (CHIR99021). Seven chemicals (7C) include a p38 inhibitor (5B202190), a JNK inhibitor (SP600125), a protein kinase C inhibitor (Go6983), a ROCK inhibitor (Y-27632), a ERK1/2 inhibitor (PD0325901) a GSK3β inhibitor (CHIR99021), and a HDAC inhibitor (VPA). Eight chemicals (8C) include a p38 inhibitor (5B202190,), a JNK inhibitor (5P600125), a protein kinase C inhibitor (Go6983), a ROCK inhibitor (Y-27632), a ERK1/2 inhibitor (PD0325901), a GSK3β inhibitor (CHIR99021), a HDAC inhibitor (VPA), and a BMPa AMPK/BMP inhibitor (Dorsomorphin).

MSCs (control MSCs derived from young men less than 40 year-old and aging MSCs derived from 40 and 69 year-old men, respectively) were cultured in the conversion medium for 6 days, without chemical treatment (control) or with chemical cocktails (six, seven or eight chemicals). The cells were later cultured in the regular medium (DMEM-LG with 10% FBS) without chemical cocktails for a further 3 days and switched to osteogenic medium for 7 days. ALP staining was performed to determine the status of osteogenic differentiation. Six chemicals (6C) includes a p38 inhibitor (SB202190), a JNK inhibitor (SP600125), a protein kinase C inhibitor (Go6983), a ROCK inhibitor (Y-27632), a ERK1/2 inhibitor (PD0325901) and a GSK3β inhibitor (CHIR99021). Seven chemicals (7C) include a p38 inhibitor (SB202190), a JNK inhibitor (SP600125), a protein kinase C inhibitor (Go6983), a ROCK inhibitor (Y-27632), a ERK1/2 inhibitor (PD0325901) a GSK3β inhibitor (CHIR99021), and a HDAC inhibitor (VPA). Eight chemicals (8C) include a p38 inhibitor (SB202190,), a JNK inhibitor (SP600125), a protein kinase C inhibitor (Go6983), a ROCK inhibitor (Y-27632), a ERK1/2 inhibitor (PD0325901), a GSK3β inhibitor (CHIR99021), a HDAC inhibitor (VPA), and a BMPa AMPK/BMP inhibitor (Dorsomorphin).

1.16 Statistical Analyses.

All statistical data are presented as the mean±standard deviation (S.D.) of at least three biological replicates. Statistically-significant differences were assessed by Student's unpaired two-tailed t-test, where p-value <0.05 was considered a significant difference.

2. Results

2.1 A Combination of Six Chemical Kinase Inhibitors with Three Growth Factors Generates iMSCs from Fibroblasts in 6 Days.

Human primary neonatal foreskin fibroblasts (CRL2097) were cultured in DMEM medium containing a chemical cocktail (6C+3GF), including six chemical kinase inhibitors i.e. a p38 inhibitor (SB202190, 10 μM), a JNK inhibitor (SP600125, 10 μM), a protein kinase C inhibitor (Go6983, 5 μM), a ROCK inhibitor (Y-27632, 5 μM), a ERK1/2 inhibitor (PD0325901, 1 μM) and a GSK3β inhibitor (CHIR99021, 3 μM) and three growth factors i.e. a leukemia inhibitory factor (LIF, 20 ng/ml), a basic fibroblast growth factor (bFGF, 8 ng/ml) and a transforming growth factor-β (TGF-β, 1 ng/ml) for 6 days in a defined and serum free medium that contain only AIbuMAX I, N2 supplement, nonessential amino acids (NEAA). FIG. 1A shows the culture process. After culture, the cells treated by the chemical cocktail were examined by flow cytometry.

As shown in FIG. 1B, the functional human MSC markers, SSEA-4 and PODXL²⁰⁻²², were significantly up-regulated in the cells after the chemical cocktail treatment (6C+3GF). To ensure the reproducibility of iMSC induction, the reprogram experiments were repeated for 10 times. Consistently, the chemical cocktail (6C+3 GF) induced iMSCs, of which 26.464.0% of cells coexpress SSEA-4 and PODXL (mean=37.62%) (FIG. 1C).

It is demonstrated that incubation of fibroblasts in the chemical cocktail (6C+3 GF) causes a proportion of the fibroblasts to reprogram into MSC-like cells (iMSCs).

2.2 iMSCs Derived from Neonatal Fibroblasts Express Traditional MSC Markers

As suggested by the Mesenchymal and Tissue Stem Cell Committee of the International Society for Cellular Therapy (ISCT), MSCs are positive for surface markers of CD105, CD73, CD44 and CD90 and negative for surface markers of CD45, CD34, CD11b, CD19 and HLA-DR.

The SSEA-4⁺PODXL⁺ iMSCs derived from human primary foreskin fibroblasts (CRL2097) were sorted and assayed by flow cytometry to determine if the iMSCs express traditional MSC markers.

As shown in FIG. 1D, the marker expression profile of the iMSCs shows positive results for CD105, CD73, CD44 and CD90 surface markers (CD105⁺, CD73⁺, CD44⁺ and CD90⁺) and negative results for CD45, CD34, CD11b, CD19 and HLA-DR surface molecules (CD45⁻, CD34⁻, CD11b⁻, CD19 and HLA-DR⁻), nearly identical to that of BMMSCs and fulfilling the MSC marker criteria defined by the Mesenchymal and Tissue Stem Cell Committee of ISCT.

2.3 the Transcriptomes of iMSCs are Similar to BMMSCs but not Fibroblasts.

The primary neonatal foreskin fibroblasts (CRL2097) and two adult dermal fibroblasts, one from a 42-year-old Caucasian female and the other from a 56-year-old Caucasian female, were induced to form iMSCs by the chemical cocktail treatment (6C+3GF). The SSEA-4⁺PODXL⁺iMSCs derived from the primary neonatal foreskin fibroblasts (CRL2097) and those derived from two adult dermal fibroblasts (DF440547, 42 year old and DF443480, 56 year old) were subjected to microarray analysis.

As shown in FIG. 1E, the cDNA expression profiles of the iMSCs, either from neonatal fibroblasts or adult fibroblasts, are more similar to those of primary human BMMSCs than those of fibroblasts.

In addition, as shown in FIG. 1F, the principal component analysis (PCA) results reveal that the iMSCs from neonatal foreskin fibroblasts and the iMSCs from adult skin fibroblasts are similar to one another, and these similarities are independent of their origins. Interestingly, iMSCs are more similar to BMMSCs than they are to their parental fibroblasts. However, in contrast, there is a clear difference between fibroblasts derived from neonatal or adult donors.

2.4 Expansion of iMSCs for at Least 8 Passages

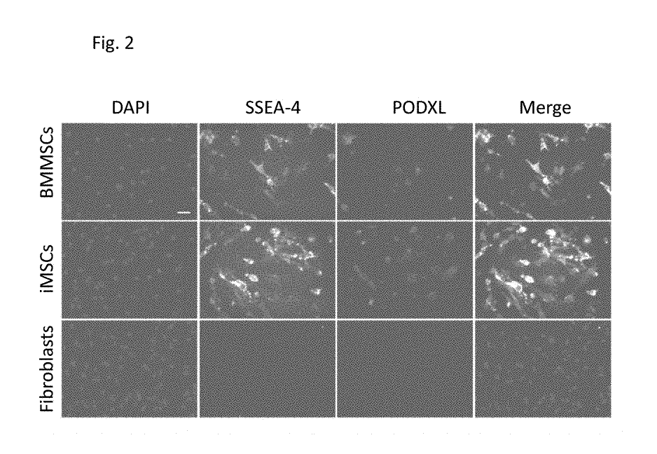

To further enrich iMSCs from the fibroblasts, the cells were sorted with SSEA-4 and PODXL. After sorting and culturing in the absence of the chemical cocktail (6C+3 GF) for 8 passages, iMSCs, like bone marrow MSCs (BMMSCs), still expressed SSEA-4 and PODXL (FIG. 2).

It is demonstrated that the iMSCs generated according to the present invention can be expanded for at least 8 passages, without losing their MSC-like features.

2.5 iMSCs Derived from Neonatal Fibroblasts are Multipotent.

To examine if the iMSCs generated according to the present invention are multipotent like primary isolated BMMSCs, we examined the ability of the iMSCs to differentiate into osteoblasts, adipocytes, and chondrocytes.

2.5.1 Osteogenesis Ability

Alkaline phosphatase (ALP) activity is required for bone formation in early osteogenesis (an early marker of osteogenesis). Alizarin Red S staining (ARS) reveals the extent of calcium deposition, which is required for bone matrix formation in late osteogenesis (a late osteogenesis marker). The iMSCs derived from neonatal fibroblasts via the chemical cocktail treatment (6C+3GF) were tested for osteogenesis ability.

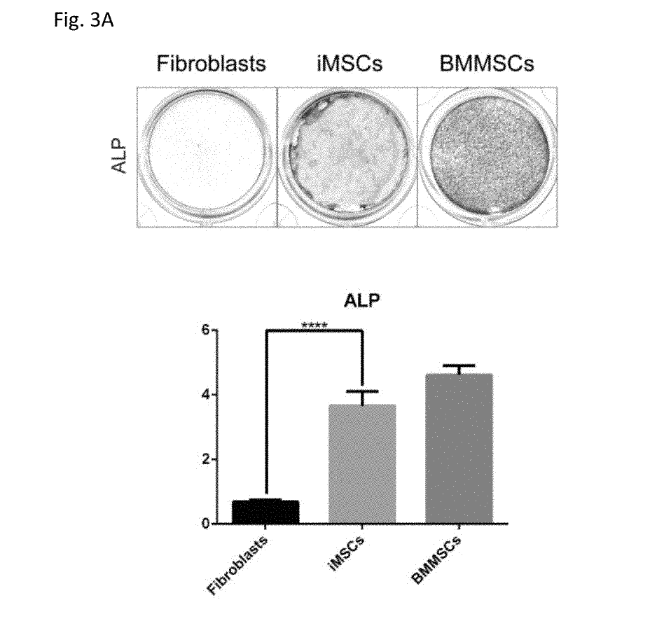

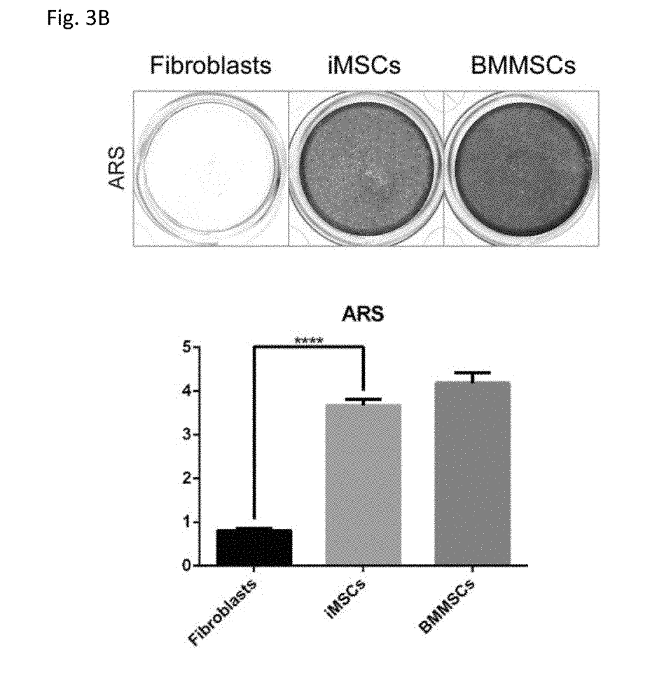

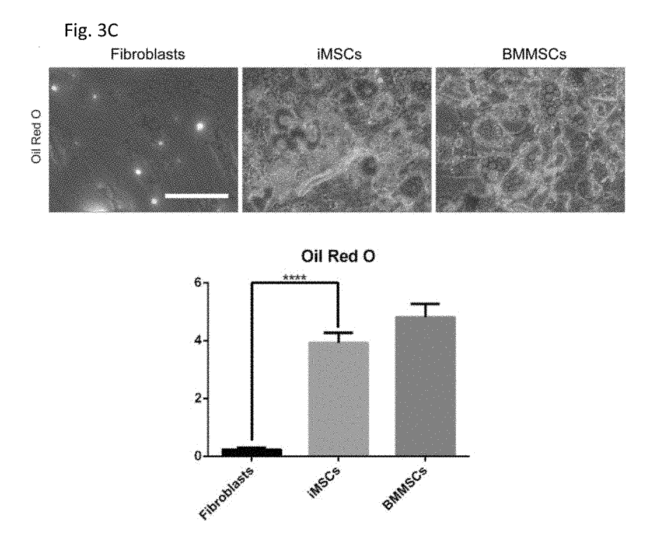

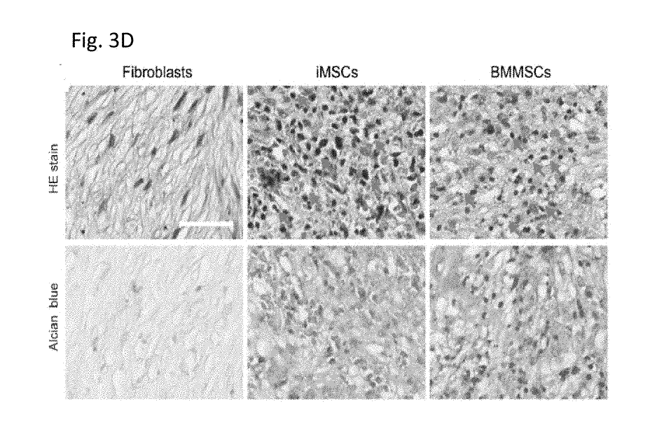

As shown in FIG. 3A and FIG. 3B, the iMSCs after osteogenic induction by incubation in osteogenic-induction medium exhibited ALP activity (an early marker of osteogenesis) at day 10 and ARS (a late osteogenesis marker) at day 21, to an extent comparable to that of primary BMMSCs; however, in contrast, primary fibroblasts showed no ALP activity and ARS results (FIG. 3A and FIG. 3B).

It is demonstrated that the iMSCs generated according to the present invention can differentiate into osteoblasts after induction.

2.5.2 Adipogenesis Ability

The iMSCs derived from neonatal fibroblasts via the chemical cocktail treatment (6C+3GF) were tested for adipogenesis ability. The iMSCs were cultured in adipocyte induction medium for 21 days and then subjected to analysis for the presence of lipid drops by Oil Red O staining.

As shown in FIG. 3C, the iMSCs, similar to BMMSCs, after adipogenic induction, exhibited a large amount of lipid drops (the middle panel and the right panel); however, in contrast, primary fibroblasts failed to generate lipid drops (the left panel).

It is demonstrated that the iMSCs according to the present invention can differentiate into adipocytes after induction.

2.5.3 Chondrogenesis Ability

The iMSCs derived from neonatal fibroblasts via the chemical cocktail treatment (6C+3GF) were tested for chondrogenesis ability. The iMSCs were cultured in chondrogenic induction medium for 21 days and then subjected to analysis for the presence of the lacunae structure of cartilage by hematoxylin-eosin (HE) staining and the presence of proteoglycans by Alcian blue staining.

As shown in FIG. 3D, the iMSCs and BMMSCs after chondrogenic induction, significantly formed the lacunae structure of cartilage and proteoglycans (the middle panel and the right panel, upper and lower); however, in contrast, primary fibroblasts failed to form the lacunae structure of cartilage and proteoglycans (the left panel, upper and lower).

It is demonstrated that the iMSCs generated according to the present invention can differentiate into chondrocytes after induction.

Given the above, it is shown that the chemical cocktail treatment (6C+3GF) is effective in inducing neonatal foreskin fibroblasts to dedifferentiate into functional iMSCs which are multipotent.

2.6 iMSCs Derived from the Adult Skin Fibroblasts are Multipotent.

To examine if iMSCs derived from various fibroblasts have similar differentiation potential, fibroblasts from two different adult donors (DF440547, 42 year old and DF443480, 56 year old) were used in the reprogramming and differentiation experiments.

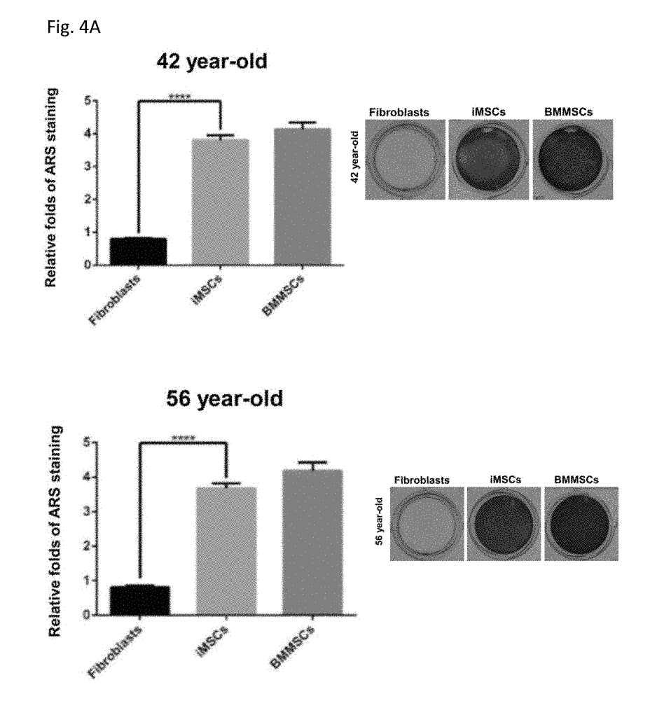

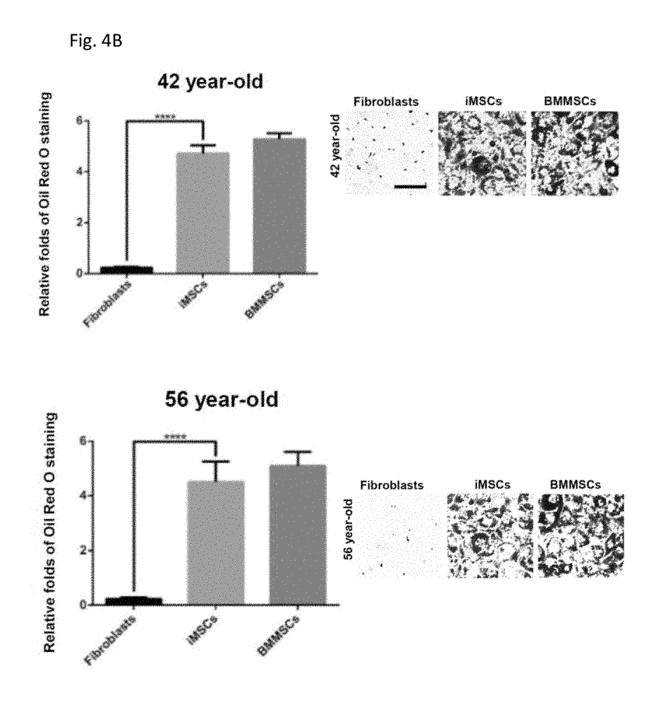

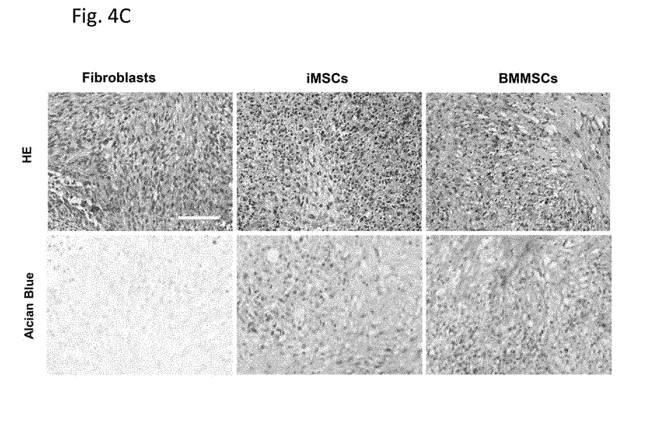

As shown in FIG. 4, the iMSCs derived from adult fibroblasts exhibited the ability to differentiate into osteoblasts (FIG. 4A), adipocytes (FIG. 4B), and chondrocytes (FIG. 4C) to a degree comparable to BMMSCs.

2.7 iMSCs Like BMMSCs Suppress the Lethality of Lipopolysaccharide Induced Lung Injury in a Mice Model.

The iMSCs generated according to the present invention were assayed for their immunomodulatory function in the mouse acute lung injury (ALI) model.

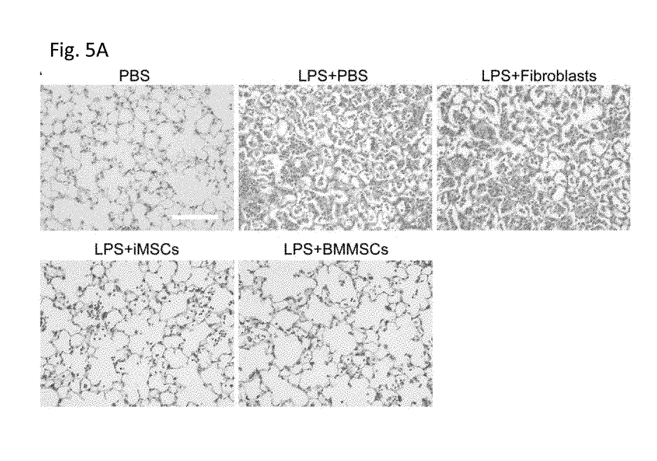

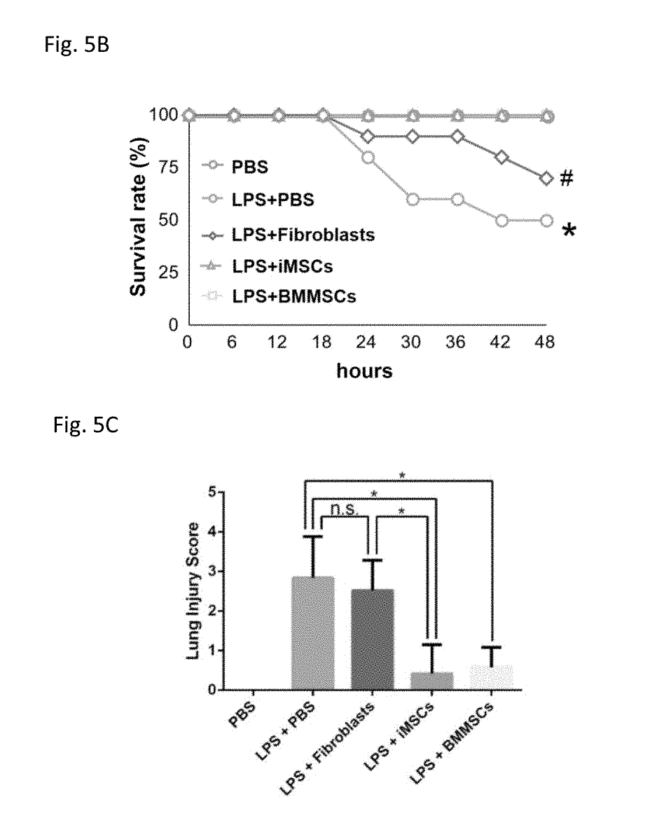

As shown in FIG. 5A, intratracheal administration of iMSCs or BMMSCs to the ALI mice 4 hours after lipopolysaccharide (LPS) treatment significantly repressed acute lung injury in the mice. Of note, as shown in FIG. 5B, all the ALI mice injected with iMSCs or BMMSCs survived; however, in contrast, around 50% death was observed in ALI mice treated with PBS or fibroblasts. As a further support as shown in FIG. 5C, ALI mice treated with iMSCs or BMMSCs exhibited lower lung injury scores, as compared with ALI mice treated with PBS or fibroblasts.

It is demonstrated that iMSCs, like BMMSCs, are therapeutically effective e.g. in inhibiting LPS-mediated ALI in vivo.

2.8 A Combination of Six Kinase Inhibitors are Effective in Generating iMSCs from Fibroblasts

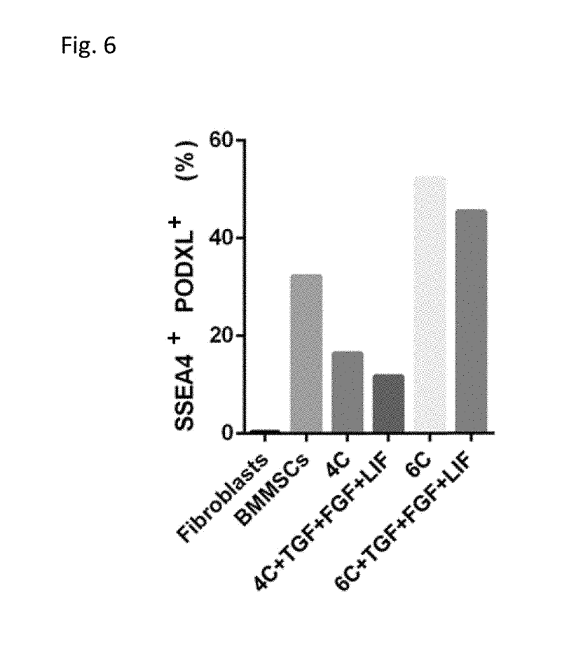

To examine which factors in the chemical cocktail (6C+3 GF) are required for iMSC generation, we try different combinations. As shown in FIG. 6, a combination of six kinase inhibitors (6C, without 3GF), including a p38 inhibitor (SB202190, 10 μM), a JNK inhibitor (SP600125, 10 μM), a PKC inhibitor (Go6983, 5 μM), a ROCK inhibitor (Y-27632, 5 μM), a ERK1/2 inhibitor (PD0325901, 1 μM) and a GSK3β inhibitor (CHIR99021, 3 μM), without the three growth factors, TGF-β, bFGF, and LIF, can effectively generate iMSCs, comparable to a combination of the six kinase inhibitors plus the three growth factors (6C+3GF). Also, a combination of the four kinase inhibitor (4C, without 3GF), including a p38 inhibitor (SB202190, 10 μM), a JNK inhibitor (SP600125, 10 μM), a protein kinase C inhibitor (Go6983, 5 μM) and a ROCK inhibitor (Y-27632, 5 μM), without the three growth factors, TGF-β, bFGF, and LIF, can effectively generate iMSCs, comparable to a combination of the four kinase inhibitors plus the three growth factors (4C+3GF), although the efficiency of the 4C combination is relatively low, when compared with the 6C combination.

Therefore, the three growth factors, TGF-β, bFGF, and LIF, can be eliminated for iMSC generation; namely a combination of the six kinase inhibitors itself (p38i+JNKi+PKCi+ROCKi+ERK1/2i+GSK3β) or a combination of the four kinase inhibitors (p38i+JNKi+PKCi+ROCKi) is effective in iMSC generation.

2.9 iMSCs Generated by Treatment of Six Kinase Inhibitors are Multipotent.

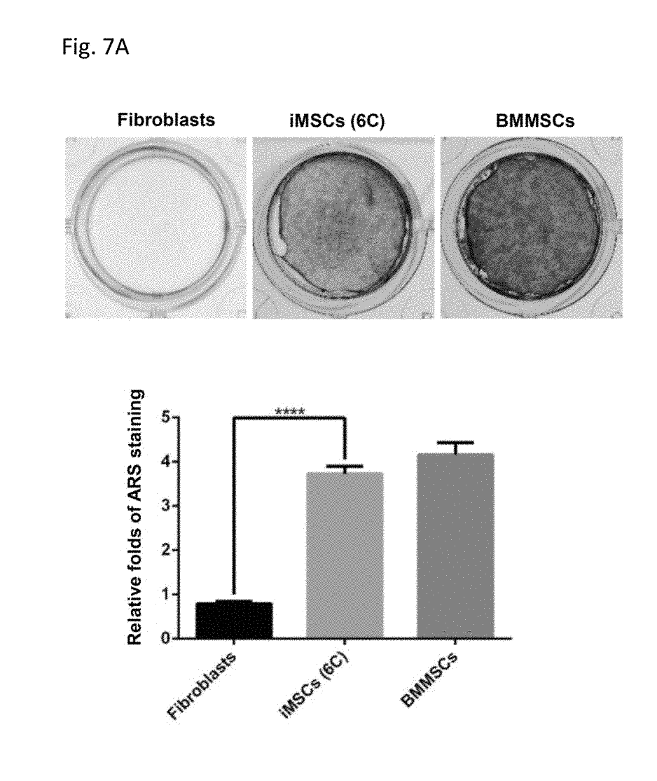

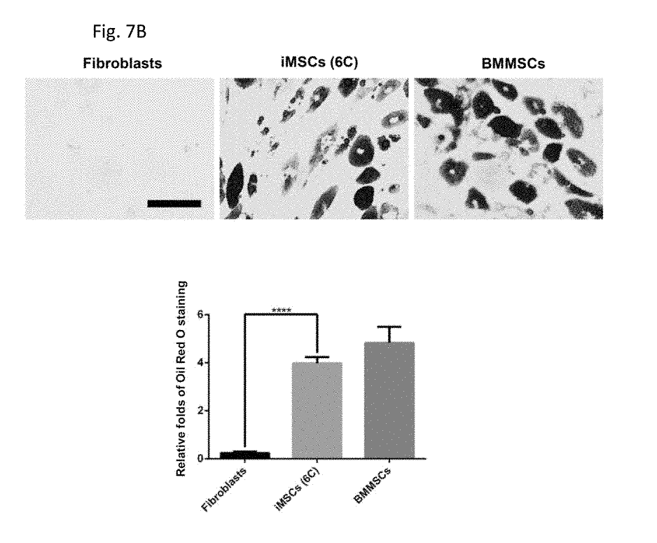

The differentiation experiments were conducted as above described. As shown in FIG. 7, the iMSCs derived from neonatal fibroblasts by treatment of six kinase inhibitors (SB202190+SP600125+Go6983+Y-27632+PD0325901+CHIR969021), without growth factors, exhibited the ability to differentiate into osteoblasts (FIG. 7A) and adipocytes (FIG. 7B) to a degree comparable to BMMSCs.

2.10 A Single Factor is Effective when Used Alone in Generating iMSCs from Fibroblasts

Human primary neonatal foreskin fibroblasts (CRL2097) were cultured in DMEM medium only (as a negative control) or in DMEM medium containing a GSK3β inhibitor (CHIR99021, 3 μM) or a protein kinase C inhibitor (Go6983, 5 μM), and the percentage of the cells converted to iMSCs, co-expressing SSEA-4 and PODXL, were determined by flow cytometer.

As shown in Table 1, a GSK3β inhibitor (CHIR99021, 3 μM) or a protein kinase C inhibitor (Go6983, 5 μM) itself is effective in iMSC generation, although the efficiency is relatively low when compared with a combination of multiple kinase inhibitors.

TABLE 1

| | Percentage of cells with both |
|---|---|
| Treatment | SSEA-4 and PODXL |
| (c) DMEM only | 0.1% |
| (1) CHIR99201 | 0.90%* |
| (2) Go6983 | 6.60%** |

Compared to DMEM only, *p < 0.05, **p < 0.001.

Some other factors or a combination were tested in the same manner. As shown in Table 2, these agents do not exhibit significant activities to generate iMSCs.

TABLE 2

| | | Percentage of cells with both |
|---|---|---|
| Treatment | | SSEA-4 and PODXL |
| (c) | DMEM only | 0.1% |
| (1) | Dorsomorphin | 0.10% |
| (2) | SB202190 | 0.10% |
| (3) | SB203580 | 0.10% |
| (4) | Fasudil | 0.20% |
| (5) | Froskolin | 0.20% |
| (6) | PP1 | 0.20% |
| (7) | SP600125 | 0.20% |
| (8) | VPA | 0.20% |
| (9) | Rapamycin | 0.20% |
| (10) | SGC0946 | 0.20% |
| (11) | Rolipram | 0.30% |
| (12) | BIX01294 | 0.30% |
| (13) | Dasatinib | 0.40% |
| (14) | GSH | 0.40% |
| (15) | PD0325901 | 0.40% |
| (16) | Repsox | 0.40% |
| (17) | SB431542 | 0.40% |
| (18) | NAC | 0.50% |
| (19) | Y27632 | 0.50% |
| (20) | VitC | 0.60% |
| (21) | SB431542 + Thiazovivin | 0.70% |
| (22) | Sodium butyrate | 0.70% |
| (23) | LY294002 | 0.80% |
| (24) | Thiazovivin | 0.80% |

Non-significant difference from 0.1% to 0.8%.

2.11 Additional Combinations of Chemicals that are Effective in Generating iMSCs from Fibroblasts

Additional combinations of chemicals were analyzed for their activities to generate iMSCs from fibroblasts and the percentage of the cells converted to iMSCs, co-expressing SSEA-4 and PODXL, were determined by flow cytometer. Table 3 shows the results.

TABLE 3

| | | Percentage of cells |
|---|---|---|
| | | with both SSEA-4 |
| Treatment | | and PODXL |
| (c) | DMEM only | 0.1% |
| (1) | Go6983^{a} + Thiazovivin^{b} | 12.00% |
| (2) | Go6983^{a} + SB431542^{b} + Thiazovivin^{b} | 13.30% |
| (3) | Go6983^{a} + Dasatinib^{b} | 13.90% |
| (4) | Go6983^{a} + CHIR99021^{a} | 14.00% |
| (5) | Go6983^{a} + VPA^{b} | 14.90% |
| (6) | Go6983^{a} + VPA^{b} + Dasatinib^{b} | 20.30% |
| (7) | Go6983^{a} + VPA^{b} + Rapamycin^{b} | 22.90% |
| (8) | Go6983^{a} + Fasudil^{b} | 24.30% |
| (9) | Go6983^{a} + VPA^{b} + BIX01294^{b} | 25.20% |
| (10) | Go6983^{a} + VPA^{b} + SGC0946^{b} | 28.30% |
| (11) | Go6983^{a} + VPA^{b} + SP600125^{b} + SB202190^{b} | 28.80% |
| (12) | Go6983^{a} + CHIR99021^{a} + | 31.30% |
| | SP600125^{b} + SB202190^{b} + Y27632^{b} + PD0325901^{b} | |
| (13) | Go6983^{a} + VPA^{b} + Froskoin^{b} | 31.40% |
| (14) | Go6983^{a} + Dorsomorphin^{b} | 36.30% |
| (15) | Go6983^{a} + CHIR99021^{a} + VPA^{b} | 40.00% |
| (16) | Go6983^{a} + CHIR99021^{a} + VPA^{b} + Y27632^{b} + PD0325901^{b} | 47.30% |
| (17) | Go6983^{a} + VPA^{b} + Dorsomorphin^{b} | 57.00% |
| (18) | Go6983^{a} + CHIR99021^{a} + VPA^{b} + Dorsomorphin^{b} | 58.00% |
| (19) | Go6983^{a} + CHIR99021^{a} + VPA^{b} + | 59.30% |
| | SP600125^{b} + SB202190^{b} + Y27632^{b} + PD0325901^{b} | |
| (20) | Go6983^{a} + CHIR99021^{a} + VPA^{b} + | 78.60% |
| | SP600125^{b} + SB202190^{b} + Y27632^{b} + | |
| | PD0325901^{b} + Dorsomorphin^{b} | |

^{a}The factor is effective in iMSC generation when used alone.
^{b}The factor is ineffective in iMSC generation when used alone.
Note:
SB202190 can be replace by another p38 inhibitor SB203580

As shown in Table 3, it is unexpected found that a combination of a PKC inhibitor C (Go6983) and a GSK3β inhibitor (CHIR99021) is effective in iMSCs generation in a synergistic manner (14.00%, treatment (4) in Table 3), when compared with either one of them when used alone (6.60% for Go6983 (treatment (2) in Table 1) and 0.9% for CHIR99021 (treatment (1) in Table 1)).

It is also unexpected found that the efficacy of Go6983 in iMSCs generation can be substantially enhanced when used in combination with one or more chemicals that are ineffective in iMSC generation when used alone. For example, a PKC inhibitor C (Go6983) when used alone can generate 6.60% of iMSCs (treatment (2) in Table 1) and a ROCK inhibitor (thiazovivin) when used alone is deemed ineffective in iMSC generation (treatment (24) in Table 2, only 0.8%); however, surprisingly, a combination of Go6983 plus thiazovivin can generate a higher percentage being 12.00% of iMSCs (treatment (1) in Table 3) in a synergistic manner. As another example, a PKC inhibitor C (Go6983) when used alone can generate 6.60% of iMSCs (treatment (2) in Table 1) and a AMPK inhibitor/BMP inhibitor (dorsomorphin) when used alone is deemed ineffective in iMSC generation (treatment (1) in Table 2, only 0.1%); however, surprisingly, a combination of Go6983 plus dorsomorphin can generate a higher percentage being 36.30% of iMSCs (treatment (14) in Table 3) in a synergistic manner; and further, the percentage can be further enhanced to 57.00% (treatment (17) in Table 3) when the combination (Go6983 plus dorsomorphin) further includes a HDAC inhibitor (VPA) that is deemed ineffective when VPA used alone in iMSC generation (treatment (8) in Table 2, only 0.2%).

Similarly, the efficacy of a combination of a PKC inhibitor C (Go6983) and a GSK3β inhibitor (CHIR99021) in iMSCs generation can be substantially enhanced when used in combination with one or more ineffective chemicals when used alone. For example, a combination of a PKC inhibitor C (Go6983) and a GSK3β inhibitor (CHIR99021) can generate 14.00% of iMSCs (treatment (4) in Table 3) and a histone deacetylase inhibitor (VPA) is deemed ineffective in iMSC generation (treatment (8) in Table 2, only 0.2%); however, surprisingly, a combination of Go6983 and CHIR99021 plus VPA can generate a higher percentage being 40.00% of iMSCs (treatment (15) in Table 3) in a synergistic manner.

As some preferred embodiments, a combination of a PKC inhibitor C (Go6983) and a GSK3β inhibitor (CHIR99021) plus a HDAC inhibitor (VPA) together with additional ineffective chemicals when used alone including a JNK inhibitor (SP600125), a p38 inhibitor (SB202190; can be replace by SB203580), a ROCK inhibitor (Y27632) and a ERK1/2 inhibitor (PD0325901) can generate a superior percentage being 59.30% of iMSCs (treatment (19) in Table 3); and the percentage can be further enhanced to 78.60% when the combination (Go6983+CHIR99021+VPA+SP600125+SB202190+Y27632+PD0325901) further includes a AMPK inhibitor/BMP inhibitor (dorsomorphin) (treatment (20) in Table 3).

2.12 Enhancement of Multipotency of MSCs

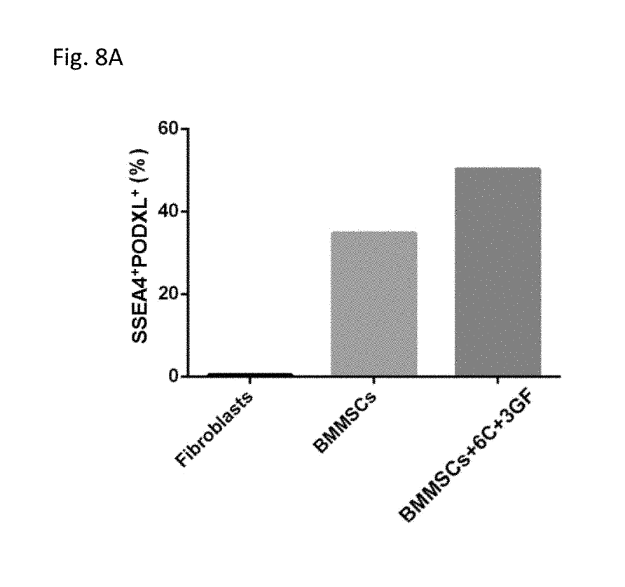

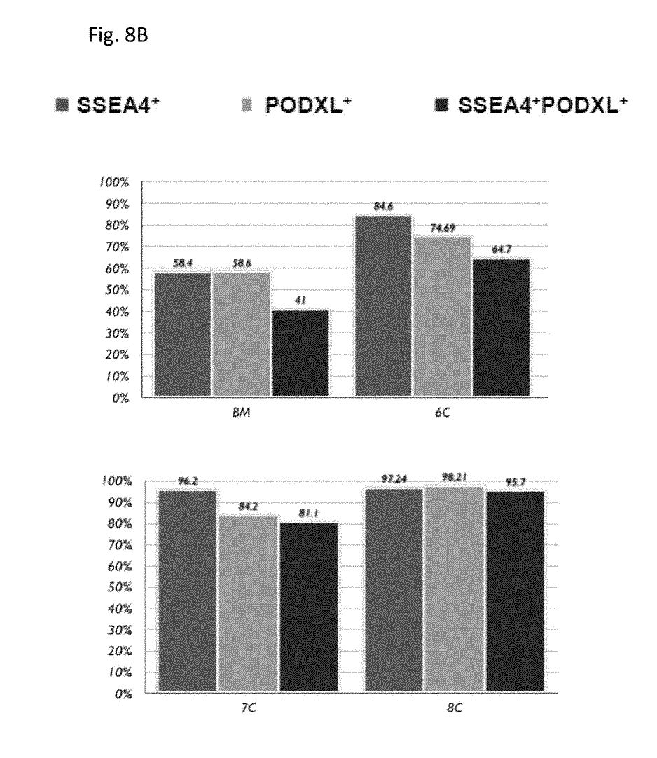

We further conducted chemical treatment of MSCs to determine the effects of chemicals in enhancing multipotency of MSCs. FIG. 8A shows that a combination of six chemical kinase inhibitors with three growth factors (6C: SB202190+SP600125+Go6983+Y27632+PD0325901+CHIR99021; 3GF: human LIF, bFGF, TGF-b) can enhance the expression of functional markers in BMMSCs from 34.8% to 50.3%, i.e. enhancing the multipotency of BMMSCs. FIG. 8B further shows that the presence of six chemicals (6C: SB202190+SP600125+Go6983+Y27632+PD0325901+CHIR99021), seven chemicals (7C: SB202190+SP600125+Go6983+Y27632+PD0325901+CHIR99021+VPA) and eight chemicals (8C: SB202190+SP600125+Go6983+Y27632+PD0325901+CHIR99021+VPA+Dorsomorphin), without growth factors, can boost the expression of functional markers from 41% to 64.7%, 81.3%, and 95.7% with 3 days of conversion. We further conducted treatment of BMMSCs with one single factor and found that one single PKC inhibitor (Go6983) can effectively increase the expression of functional markers of BMSCs. We further conducted treatment of BMMSCs with four (4) chemicals (4C: Go6983+CHIR99021+VPA+Dorsomorphin) and found that the 4C treatment also can effectively increase the expression of functional markers of BMSCs.

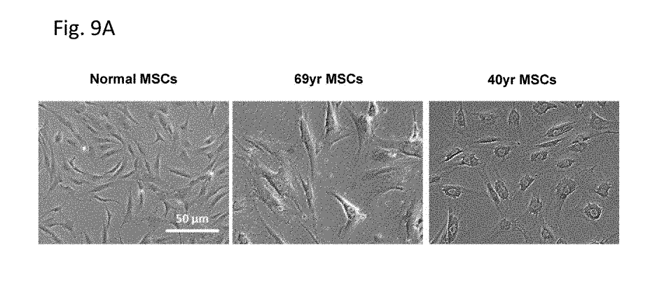

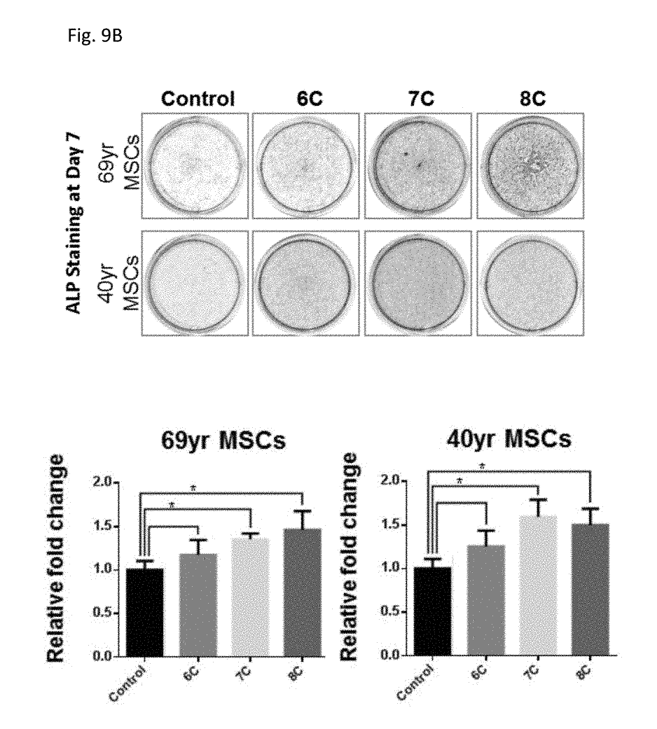

Moreover, the osteogenesis ability is also enhanced in aging MSCs by the chemical treatment of the present invention. As shown FIG. 9A, two primary aging MSCs were isolated from the donors. Comparing to the healthy control which is less than 35 year old, the MSCs from 40 year old donor and 69 year old donor shows aging phenotype. The cells lost their spindle shape morphology and the granularity increased. As shown in FIG. 9B, after treating with six (6) chemicals (6C: SB202190+SP600125+Go6983+Y27632+PD0325901+CHIR99021), seven (7) chemicals (7C: SB202190+SP600125+Go6983+Y27632+PD0325901+CHIR99021+VPA) and eight (8) chemicals (8C: SB202190+SP600125+Go6983+Y27632+PD0325901+CHIR99021+VPA+Dorsomorphin), the osteogenesis increased. It demonstrated that the chemical cocktails can improve the multipotency of aging MSCs.

3. Summary

In summary, we reported, for the first time, an approach to generate iMSCs from fibroblasts by using chemical treatment with a PKC inhibitor C and/or a GSK3β inhibitor, optionally in combination with one or more auxiliary agent e.g. ineffective chemicals when used alone. The approach does not need to use serum for cell culture (i.e. serum-free) which is suitable for clinical applications. The approach also can be xeno-free. Further, the approach does not require steps that may lead to insertional mutagenesis e.g. virus infection or plasmid transfection.

In certain embodiments, the conversion rate of iMSCs from fibroblasts according to the present invention can be higher than about 1% and particular can reach to about 80%. The iMSCs generated according to the present invention exhibit MSC's features, including expression of SSEA-4 and PODXL and other MSC markers (CD105⁺, CD73⁺, CD44⁺and CD90⁺), multipotent activities to differentiate into osteoblasts, adipocytes and chondrocytes, for example, and therapeutic effects at least in treating endotoxin-induced ALI animals. The approach of the present invention is effective in generating functional iMSCs and suitable for regenerative medicine in treating multiple diseases.

In addition, the chemical treatment as described herein can also improve the MSC's functional characteristics, such as the activities in expansion, clonogenicity and/or differentiation, which is advantageous in cell therapy.

### REFERENCES

1. Haynesworth, S. E., Goshima, J., Goldberg, V. M. & Caplan, A. I. Characterization of cells with osteogenic potential from human marrow. Bone 13, 81-88 (1992).
2. Pittenger, M. F. et al. Multilineage potential of adult human mesenchymal stem cells. Science 284, 143-147 (1999).
3. Kfoury, Y. & Scadden, D. T. Mesenchymal cell contributions to the stem cell niche. Cell Stem Cell 16, 239-253, doi:10.1016/j.stem.2015.02.019 (2015).
4. Wang, Y., Chen, X., Cao, W. & Shi, Y. Plasticity of mesenchymal stem cells in immunomodulation: pathological and therapeutic implications. Nat Immunol 15, 1009-1016, doi:10.1038/ni.3002 (2014).
5. Karp, J. M. & Leng Teo, G. S. Mesenchymal stem cell homing: the devil is in the details. Cell Stem Cell 4, 206-216, doi:10.1016/j.stem.2009.02.001 (2009).
6. Fang, X. et al. Human Mesenchymal Stem (Stromal) Cells Promote the Resolution of Acute Lung Injury in Part through Lipoxin A4. J Immunol 195, 875-881, doi:10.4049/jimmunol.1500244 (2015).
7. Le Blanc, K. et al. Treatment of severe acute graft-versus-host disease with third party haploidentical mesenchymal stem cells. Lancet 363, 1439-1441, doi:10.1016/S0140-6736(04)16104-7 (2004).
8. Duijvestein, M. et al. Autologous bone marrow-derived mesenchymal stromal cell treatment for refractory luminal Crohn's disease: results of a phase I study. Gut 59, 1662-1669, doi:10.1136/gut.2010.215152 (2010).
9. Ankrum, J. & Karp, J. M. Mesenchymal stem cell therapy: Two steps forward, one step back. Trends Mol Med 16, 203-209, doi:10.1016/j.molmed.2010.02.005 (2010).
10. Mazzini, L. et al. Stem cell therapy in amyotrophic lateral sclerosis: a methodological approach in humans. Amyotroph Lateral Scler Other Motor Neuron Disord 4, 158-161 (2003).
11. Chen, S. L. et al. Effect on left ventricular function of intracoronary transplantation of autologous bone marrow mesenchymal stem cell in patients with acute myocardial infarction. Am J Cardiol 94, 92-95, doi:10.1016/j.amjcard.2004.03.034 (2004).
12. Horwitz, E. M. et al. Isolated allogeneic bone marrow-derived mesenchymal cells engraft and stimulate growth in children with osteogenesis imperfecta: Implications for cell therapy of bone. Proc Natl Acad Sci USA 99, 8932-8937, doi:10.1073/pnas.132252399 (2002).
13. Field, R. E., Buchanan, J. A., Copplemans, M. G. & Aichroth, P. M. Bone-marrow transplantation in Hurler's syndrome. Effect on skeletal development. J Bone Joint Surg Br 76, 975-981 (1994).
14. Trounson, A. & McDonald, C. Stem Cell Therapies in Clinical Trials: Progress and Challenges. Cell Stem Cell 17, 11-22, doi:10.1016/j.stem.2015.06.007 (2015).
15. Gao, F. et al. Mesenchymal stem cells and immunomodulation: current status and future prospects. Cell Death Dis 7, e2062, doi:10.1038/cddis.2015.327 (2016).
16. de Girolamo, L. et al. Mesenchymal stem/stromal cells: a new “cells as drugs” paradigm. Efficacy and critical aspects in cell therapy. Curr Pharm Des 19, 2459-2473 (2013).
17. Galotto, M. et al. Stromal damage as consequence of high-dose chemo/radiotherapy in bone marrow transplant recipients. Exp Hematol 27, 1460-1466 (1999).
18. Bosi, A. & Bartolozzi, B. Safety of bone marrow stem cell donation: a review. Transplant Proc 42, 2192-2194, doi:10.1016/j.transproceed.2010.05.029 (2010).
19. Vangipuram, M., Ting, D., Kim, S., Diaz, R. & Schule, B. Skin punch biopsy explant culture for derivation of primary human fibroblasts. J Vis Exp, e3779, doi:10.3791/3779 (2013).
20. Gang, E. J., Bosnakovski, D., Figueiredo, C. A., Visser, J. W. & Perlingeiro, R. C. SSEA-4 identifies mesenchymal stem cells from bone marrow. Blood 109, 1743-1751, doi:10.1182/blood-2005-11-010504 (2007).
21. Rosu-Myles, M. et al. The globoseries glycosphingolipid SSEA-4 is a marker of bone marrow-derived clonal multipotent stromal cells in vitro and in vivo. Stem Cells Dev 22, 1387-1397, doi:10.1089/scd.2012.0547 (2013).
22. Lee, R. H. et al. The CD34-like protein PODXL and alpha6-integrin (CD49f) identify early progenitor MSCs with increased clonogenicity and migration to infarcted heart in mice. Blood 113, 816-826, doi:10.1182/blood-2007-12-128702 (2009).
23. Gafni, O. et al. Derivation of novel human ground state naive pluripotent stem cells. Nature 504, 282-286, doi:10.1038/nature12745 (2013).
24. Zhang, W. & Liu, H. T. MAPK signal pathways in the regulation of cell proliferation in mammalian cells. Cell Res 12, 9-18, doi:10.1038/sj.cr.7290105 (2002).
25. Fang, R. et al. Generation of naive induced pluripotent stem cells from rhesus monkey fibroblasts. Cell Stem Cell 15, 488-496, doi:10.1016/j.stem.2014.09.004 (2014).
26. Chandrakanthan, V. et al. PDGF-AB and 5-Azacytidine induce conversion of somatic cells into tissue-regenerative multipotent stem cells. Proceedings of the National Academy of Sciences 113(16):E2306-15. doi: 10.1073/pnas.1518244113 (2016).

What is claimed is:

1. A method of generating induced mesenchymal stem cells (iMSCs), comprising culturing skin cells in a condition which allows a proportion of the skin cells to dedifferentiate into iMSCs, wherein the condition comprises a culture medium which comprises a protein kinase C (PKC) inhibitor and/or a glycogen synthase kinase 3 beta (GSK3β) inhibitor.

2. The method of claim 1, wherein the skin cells are fibroblasts.

3. The method of claim 1, wherein the culture medium further comprises an auxiliary agent selected from the group consisting of a p38 inhibitor, a c-jun N terminal kinase (JNK) inhibitor, a Rho-associated protein kinase (ROCK) inhibitor, an extracellular regulated kinase (ERK) inhibitor, an AMP-activated protein kinase (AMPK) inhibitor, a Src tyrosine kinase inhibitor, an anaplastic lymphoma kinase (ALK) inhibitor, a phosphoinositide 3-kinase inhibitor (PI3K) inhibitor, a cyclic adenosine monophosphate (cAMP) activator, a histone deacetylase (HDAC) inhibitor, an antioxidant, a antioxidant, a tumor growth factor beta (TGFβ) inhibitor, a molecular target of rapamycin (mTOR) inhibitor, a G9a methyltransferase inhibitor, a DOTIL inhibitor and any combination thereof.

4. The method of claim 3, wherein the culture medium comprises a combination selected from the group consisting of:
(1) a combination of a PKC inhibitor and a ROCK inhibitor;
(2) a combination of a PKC inhibitor, a ALK inhibitor and a ROCK inhibitor;
(3) a combination of a PKC inhibitor and a Src family tyrosine kinase inhibitor;
(4) a combination of a PKC inhibitor and a GSK3β inhibitor;
(5) a combination of a PKC inhibitor and a HDAC inhibitor;
(6) a combination of a PKC inhibitor, a HDAC inhibitor and a Src tyrosine kinase inhibitor;
(7) a combination of a PKC inhibitor, a HDAC inhibitor and a target of rapamycin (mTOR) inhibitor;
(8) a combination of a PKC inhibitor and a cAMP activator;
(9) a combination of a PKC inhibitor, a HDAC inhibitor and a G9a methyltransferase inhibitor;
(10) a combination of a PKC inhibitor, a HDAC inhibitor and a DOT1L inhibitor;
(11) a combination of a PKC inhibitor, a HDAC inhibitor, a JNK inhibitor and a p38 inhibitor;
(12) a combination of a PKC inhibitor, a GSK3β inhibitor, a JNK inhibitor, a p38 inhibitor, a ROCK inhibitor and a ERK inhibitor;
(13) a combination of a PKC inhibitor, a HDAC inhibitor and a cAMP activator;
(14) a combination of a PKC inhibitor and an AMPK/BMP inhibitor;
(15) a combination of a PKC inhibitor, a GSK3β inhibitor and a HDAC inhibitor;
(16) a combination of a PKC inhibitor, a GSK3β inhibitor, a HDAC inhibitor, a ROCK inhibitor and an ERK inhibitor;
(17) a combination of a PKC inhibitor, a HDAC inhibitor and an AMPK/BMP inhibitor;
(18) a combination of a PKC inhibitor, a GSK3β inhibitor, a HDAC inhibitor and a AMPK inhibitor/BMP inhibitor
(19) a combination of a PKC inhibitor, a GSK3β inhibitor, a HDAC inhibitor, a JNK inhibitor, a p38 inhibitor, a ROCK inhibitor and a ERK inhibitor; and
(20) a combination of a PKC inhibitor, a GSK3β inhibitor, a HDAC inhibitor, a JNK inhibitor, a p38 inhibitor, a ROCK inhibitor, a ERK inhibitor, and a AMPK inhibitor/BMP inhibitor.

5. The method of claim 3, wherein the culture medium comprises a combination selected from the group consisting of:
(4) a combination of a PKC inhibitor and a GSK3β inhibitor;
(12) a combination of a PKC inhibitor, a GSK3β inhibitor, a JNK inhibitor, a p38 inhibitor, a ROCK inhibitor and a ERK inhibitor;
(15) a combination of a PKC inhibitor, a GSK3β inhibitor and a HDAC inhibitor;
(16) a combination of a PKC inhibitor, a GSK3β inhibitor, a HDAC inhibitor, a ROCK inhibitor and an ERK inhibitor;
(18) a combination of a PKC inhibitor, a GSK3β inhibitor, a HDAC inhibitor and a AMPK inhibitor BMP inhibitor;
(19) a combination of a PKC inhibitor, a GSK3β inhibitor, a HDAC inhibitor, a JNK inhibitor, a p38 inhibitor, a ROCK inhibitor and a ERK inhibitor; and
(20) a combination of a PKC inhibitor, a GSK3β inhibitor, a HDAC inhibitor, a JNK inhibitor, a p38 inhibitor, a ROCK inhibitor, a ERK inhibitor, and a AMPK/BMP inhibitor.

6. The method of claim 3, wherein the culture medium comprises a combination selected from the group consisting of:
(5) a combination of a PKC inhibitor and a HDAC inhibitor;
(6) a combination of a PKC inhibitor, a HDAC inhibitor and a Src tyrosine kinase inhibitor;
(7) a combination of a PKC inhibitor, a HDAC inhibitor and a target of rapamycin (mTOR) inhibitor;
(9) a combination of a PKC inhibitor, a HDAC inhibitor and a G9a methyltransferase inhibitor;
(10) a combination of a PKC inhibitor, a HDAC inhibitor and a DOT1L inhibitor;
(11) a combination of a PKC inhibitor, a HDAC inhibitor, a JNK inhibitor and a p38 inhibitor;
(13) a combination of a PKC inhibitor, a HDAC inhibitor and a cAMP activator;
(15) a combination of a PKC inhibitor, a GSK3β inhibitor and a HDAC inhibitor;
(16) a combination of a PKC inhibitor, a GSK3β inhibitor, a HDAC inhibitor, a ROCK inhibitor and an ERK inhibitor;
(17) a combination of a PKC inhibitor, a HDAC inhibitor and an AMPK/BMP inhibitor;
(18) a combination of a PKC inhibitor, a GSK3β inhibitor, a HDAC inhibitor and a AMPK inhibitor BMP inhibitor;
(19) a combination of a PKC inhibitor, a GSK3β inhibitor, a HDAC inhibitor, a JNK inhibitor, a p38 inhibitor, a ROCK inhibitor and a ERK inhibitor; and
(20) a combination of a PKC inhibitor, a GSK3β inhibitor, a HDAC inhibitor, a JNK inhibitor, a p38 inhibitor, a ROCK inhibitor, a ERK inhibitor, and a AMPK/BMP inhibitor.

7. The method of claim 3, wherein the culture medium comprises a combination selected from the group consisting of:
(15) a combination of a PKC inhibitor, a GSK3β inhibitor and a HDAC inhibitor;
(16) a combination of a PKC inhibitor, a GSK3β inhibitor, a HDAC inhibitor, a ROCK inhibitor and an ERK inhibitor;
(18) a combination of a PKC inhibitor, a GSK3β inhibitor, a HDAC inhibitor and a AMPK inhibitor BMP inhibitor;
(19) a combination of a PKC inhibitor, a GSK3β inhibitor, a HDAC inhibitor, a JNK inhibitor, a p38 inhibitor, a ROCK inhibitor and a ERK inhibitor; and
(20) a combination of a PKC inhibitor, a GSK3β inhibitor, a HDAC inhibitor, a JNK inhibitor, a p38 inhibitor, a ROCK inhibitor, a ERK inhibitor, and a AMPK/BMP inhibitor.

8. The method of claim 3, wherein the culture medium comprises a combination selected from the group consisting of:
(14) a combination of a PKC inhibitor and an AMPK/BMP inhibitor;
(17) a combination of a PKC inhibitor, a HDAC inhibitor and an AMPK/BMP inhibitor;
(18) a combination of a PKC inhibitor, a GSK3β inhibitor, a HDAC inhibitor and a AMPK inhibitor/BMP inhibitor;
(20) a combination of a PKC inhibitor, a GSK3β inhibitor, a HDAC inhibitor, a JNK inhibitor, a p38 inhibitor, a ROCK inhibitor, a ERK inhibitor, and a AMPK/BMP inhibitor.

9. The method of claim 3, wherein the culture medium comprises a combination selected from the group consisting of:
(1) a combination of a PKC inhibitor and a ROCK inhibitor;
(2) a combination of a PKC inhibitor, a ALK inhibitor and a ROCK inhibitor;
(12) a combination of a PKC inhibitor, a GSK3β inhibitor, a JNK inhibitor, a p38 inhibitor, a ROCK inhibitor and a ERK inhibitor;
(16) a combination of a PKC inhibitor, a GSK3β inhibitor, a HDAC inhibitor, a ROCK inhibitor and an ERK inhibitor and;
(19) a combination of a PKC inhibitor, a GSK3β inhibitor, a HDAC inhibitor, a JNK inhibitor, a p38 inhibitor, a ROCK inhibitor and a ERK inhibitor; and
(20) a combination of a PKC inhibitor, a GSK3β inhibitor, a HDAC inhibitor, a JNK inhibitor, a p38 inhibitor, a ROCK inhibitor, a ERK inhibitor, and a AMPK/BMP inhibitor.

10. The method of claim 3, wherein the culture medium comprises a combination selected from the group consisting of:
(12) a combination of a PKC inhibitor, a GSK3β inhibitor, a JNK inhibitor, a p38 inhibitor, a ROCK inhibitor and a ERK inhibitor;
(19) a combination of a PKC inhibitor, a GSK3β inhibitor, a HDAC inhibitor, a JNK inhibitor, a p38 inhibitor, a ROCK inhibitor and a ERK inhibitor; and
(20) a combination of a PKC inhibitor, a GSK3β inhibitor, a HDAC inhibitor, a JNK inhibitor, a p38 inhibitor, a ROCK inhibitor, a ERK inhibitor, and a AMPK/BMP inhibitor.

11. The method of claim 1, wherein the inhibitor(s) is/are small molecule(s).

12. The method of claim 3, wherein the auxiliary agent is a small molecule.

13. The method of claim 3, wherein the culture medium comprises a combination selected from the group consisting of:
(1) a combination of Go6983 and Thiazovivin;
(2) a combination of Go6983, SB431542 and Thiazovivin;
(3) a combination of Go6983 and Dasatinib;
(4) a combination of Go6983 and CHIR99021;
(5) a combination of Go6983 and VPA;
(6) a combination of Go6983, VPA and Dasatinib;
(7) a combination of Go6983, VPA and Rapamycin;
(8) a combination of Go6983 and Fasudil;
(9) a combination of Go6983, VPA and BIX01294;
(10) a combination of Go6983, VPA and SGC0946;
(11) a combination of Go6983, VPA, SP600125 and SB202190;
(11-1) a combination of Go6983, VPA, SP600125 and SB203580;
(12) a combination of Go6983, CHIR99021, SP600125, SB202190, Y27632 and PD0325901;
(12-1) a combination of Go6983, CHIR99021, SP600125, SB203580, Y27632 and PD0325901;
(13) a combination of Go6983, VPA and Froskoin;
(14) a combination of Go6983 and Dorsomorphin;
(15) a combination of Go6983, CHIR99021 and VPA;
(16) a combination of Go6983, CHIR99021, VPA, Y27632 and PD0325901;
(17) a combination of Go6983, VPA and Dorsomorphin;
(18) a combination of Go6983, CHIR99021, VPA and Dorsomorphin;
(19) a combination of Go6983, CHIR99021, VPA, SP600125, SB202190, Y27632 and PD0325901;
(19-1) a combination of Go6983, CHIR99021, VPA, SP600125, SB203580, Y27632 and PD0325901;
(20) a combination of Go6983, CHIR99021, VPA, SP600125, SB202190, Y27632, PD0325901, and Dorsomorphin, and
(20-1) a combination of Go6983, CHIR99021, VPA, SP600125, SB203580, Y27632, PD0325901, and Dorsomorphin.
(21) Go6983.
(22) CHIR99021.

14. The method of claim 1, wherein the skin cells are human cells.

15. The method of claim 2, wherein the fibroblasts are neonatal fibroblasts or adult fibroblasts.

16. The method of claim 1, wherein the skin cells are cultured in the culture medium for at least 1 day or more.

17. The method of claim 1, wherein the culture medium is serum free.

18. The method of claim 1, wherein about 1 to 80% of the skin cells are dedifferentiated into iMSCs.

19. The method of claim 1, further comprising isolating the iMSCs from the cell culture to obtain an isolated population of iMSCs.

20. A method of producing differentiated somatic cells, comprising subjecting iMSCs derived from somatic cells via treatment with a protein kinase C (PKC) inhibitor and/or a glycogen synthase kinase 3 beta (GSK3β) inhibitor, and optionally one or more auxiliary agents, to a condition suitable for differentiation, thereby producing specific somatic cells.

21. The method of claim 20, wherein the specific somatic cells are selected from the group consisting of fibroblasts, adipocytes, chondrocytes, osteoblasts, osteocytes, myoblasts, neurons, beta islet cells, hepatocytes, cardiomyocytes and neural stem cells.

22. A method for treating a disease or disorder, comprising administering a therapeutically effective amount of iMSCs which are derived from fibroblasts via treatment with a protein kinase C (PKC) inhibitor and/or a glycogen synthase kinase 3 beta (GSK3β) inhibitor, and optionally one or more auxiliary agents, to a subject in need of such treatment.

23. The method of claim 22, wherein the disease or disorder is selected from the group consisting of acute lung injury (ALI), graft-versus-host Disease, Crohn's disease, type 1 diabetes mellitus, diabetic wounds, multiple sclerosis, neurological diseases (spinal cord Injury, Parkinson's disease, Alzheimer's disease, amyotrophic lateral sclerosis, diabetic peripheral neuropathy, epilepsy, schizophrenia, autism), cardiovascular diseases (myocardial infraction, ischemic heart disease, chronic heart failure, coronary artery disease, dilated cardiomyopathy peripheral vascular diseases, non-ischemic dilated cardiomyopathy), osteogenesis imperfecta, ulcerative colitis, stem cell engraftment, cirrhosis, fractures, cartilage injury, kidney transplant, renal failure, osteoarthritis, acute respiratory distress syndrome, Sjögren's syndrome (pSS), systematic sclerdomerma, Duchenne muscular dystrophy, cancers, degenerative disc disease, arthroscopic rotator cuff repair, anemia, critical limb ischemia, neuromyelitis optica spectrum disorders, subclinical rejection of organ tranpinatation, maxillary cyst, atherosclerosis, premature ovarian failure, anterior cruciate ligament injury, articular chondral defect, Kienböck's disease, sepeis/septic shock, perianal fistula, osteonecrosis, pseudoarthrosis, delayed graft function, focal segmental glomerulosclerosis, chronic obstructive pulmonary disease, osteochondritis, rheumatoid arthritis, dysphonia, osteonecrosis, drug-induced neutropenia, brain injuries, burn wound, acute kidney injury, breast reconstruction, liver failure, liver cirrhosis, foreign body reaction, inflammation, effusion (L) knee, skin ulcer, recto-vaginal fistula, dystrophic epidermolysis bullosa, osteoporosis, local feminine stress urinary incontinence treatment (HULPURO), retinal disease, macular degeneration, hereditary retinal dystrophy, optic nerve disease, glaucoma, hip arthroplasty, cerebral palsy, male infertility, arthrodesis, Romberg's disease, ankylosing spondylitis, uremia, chronic meniscal injury, cutaneous photoaging, emphysema, bronchopulmonary dysplasia, fecallncontinence, idiopathic pulmonary fibrosis, autoimmune hepatitis, biliary cirrhosis, spondyloarthrosis, epidermolysis bullosa, asthma, xerostomia, dementia, recovery of medial meniscectomy, progressive supranuclear palsy, psoriasis vulgaris, CMV infection, rotator cuff disease, cytopenia, myelodysplastic syndromes, Peyronie's-Disease, limbus corneae insufficiency syndrome, Romberg's disease, liver regeneration, refractory-systemic lupus erythematosus, ulcerative colitis, paraquat Poisoning, pneumonia, emphysema, aging-frailty, lung transplantation, bone cyst, cerebral adrenoleukodystrophy, erectile dysfunction, intervertebral disc disease, lipodystrophies, Buerger's-disease, hemophilia, Wilson's disease, bronchiectasis, retinitis pigmentosa, cerebellar Ataxia, sweat gland diseases, systemic lupus erythematosus, Devic's Syndrome, cleft lip and palate, Sjogren's Syndrome and Hurler's syndrome.

24. A method of improving functional characteristics of MSCs, comprising treating the MSCs with a protein kinase C (PKC) inhibitor and/or a glycogen synthase kinase 3 beta (GSK3β) inhibitor, and optionally one or more auxiliary agents.

25. The method of claim 24, wherein the one or more auxiliary agents are selected from the group consisting of a p38 inhibitor, a c-jun N terminal kinase (JNK) inhibitor, a Rho-associated protein kinase (ROCK) inhibitor, an extracellular regulated kinase (ERK) inhibitor, an AMP-activated protein kinase (AMPK) inhibitor, a Src tyrosine kinase inhibitor, an anaplastic lymphoma kinase (ALK) inhibitor, a phosphoinositide 3-kinase inhibitor (PI3K) inhibitor, a cyclic adenosine monophosphate (cAMP) activator, a histone deacetylase (HDAC) inhibitor, an antioxidant, a antioxidant, a tumor growth factor beta (TGFβ) inhibitor, a molecular target of rapamycin (mTOR) inhibitor, a G9a methyltransferase inhibitor, a DOTIL inhibitor and any combination thereof.

26. The method of claim 24, wherein the functional characteristics of MSCs include activities in expansion, clonogenicity and/or differentiation.
